(19) United States
(12) Patent Application Publication
(10) Pub. No.: US 2021/0170072 A1
(43) Pub. Date: Jun. 10, 2021

(54) ATMOSPHERE-BREATHING REFILLABLE BIPHASIC DEVICE FOR CELL REPLACEMENT THERAPY

(71) Applicant: Cornell University, Ithaca, NY (US)

(72) Inventors: Duo An, San Mateo, CA (US); Longhai Wang, Ithaca, NY (US); Alexander Ernst, Ithaca, NY (US); James Flanders, Ithaca, NY (US); Minglin Ma, Ithaca, NY (US)

(21) Appl. No.: 17/113,897

(22) Filed: Dec. 7, 2020

Related U.S. Application Data
(60) Provisional application No. 62/944,169, filed on Dec. 5, 2019.

(51) Int. Cl. A61L 27/38; A61L 27/16; A61L 27/06; A61L 27/18; A61L 27/56; A61L 27/52; A61L 27/54; A61P 3/10; A61M 5/14

(52) U.S. Cl. CPC A61L 27/3804; A61M 2202/097; A61L 27/06; A61L 27/18; A61L 27/56; A61L 27/52; A61L 27/54; A61P 3/10; A61M 5/14; A61L 2300/64; A61L 2400/18; A61M 2205/04; A61M 2202/09; A61M 2202/07; A61L 27/16

(57) ABSTRACT

The present application relates cell replacement devices, comprising a frame cap and a frame base, where the frame cap includes a first connecting member and one or more ports that traverse a thickness of the frame cap. The frame base of the cell replacement device includes one or more walls defining an interior chamber, defining a first opening to the interior chamber on one side of the frame base, and defining a second opening to the interior chamber on another side of the frame base. The first opening of the frame base is configured to receive the frame cap, and the frame base further includes a second connecting member constructed to connect with the first connecting member. The frame base further comprises a mesh disposed adjacent the second opening.

This application claims the benefit of U.S. Provisional Patent Application Ser. No. 62/944,169, filed Dec. 5, 2019, which is hereby incorporated by reference in its entirety.

### FIELD

The present application relates to cell replacement devices and methods of delivering cells or a therapeutic agent produced by said cells to a subject in need thereof.

### BACKGROUND

Cell replacement therapies take advantage of the dynamic responsiveness and activity of cells and promise to improve treatment for a number of pathologies including endocrine disorders and hormone deficient diseases (Fischbach, et al., “Cell-based Therapeutics: the Next Pillar of Medicine,” Sci. Transl. Med. 5:179p57 (2013); Lee et al., “Cell Transplantation of Endocrine Disorders,” Adv. Drug Del. Rev 42:103-20 (2000); Ernst et., “Nanotechnology in Cell Replacement Therapies for Type 1 Diabetes,” Adv. Drug Del. Rev. (2019)). The encapsulation and protection of the transplanted cells from the host immune system via a semipermeable material or device is required, in many cases, to localize the cells and prevent immune rejection (Farina et al., “Cell Encapsulation: Overcoming Barriers in Cell Transplantation in Diabetes and Beyond,” Adv. Drug Del. Rev. (2019)). While cell encapsulation can overcome several problems of cell replacement therapies such as obviating the co-administration of immunosuppressive drugs, critical limitations barring clinical translation remain.

Primary among these limitations is the lack of adequate oxygen supply to the encapsulated cells (Colton, “Oxygen Supply to Encapsulated Therapeutic Cells,” Adv. Drug Del. Rev. 93:67-68 (2014)). Immuno-isolation by polymer encapsulation necessarily dissociates the cells from the host vasculature and thus the cells rely on oxygen delivery by passive diffusion over distances of hundreds of microns (depending on device design). Moreover, dissolved oxygen levels are considerably lower in common transplantation sites (e.g. the subcutaneous space) between 8-35 mmHg (Carreau et al., “Why is the Partial Oxygen Pressure of Human Tissues a Crucial Parameter? Small Molecules and Hypoxia,” J. Cell. Mol. Med. 15:1239-53 (2011)) as compared to arterial oxygen tensions near 100 mmHg (Bochenek, et al., “Alginate Encapsulation as Long-term Immune Protection of Allogeneic Pancreatic Islet Cells Transplanted into the Omental Bursa of Macaques,” Nat. Biomed. Eng. 2:810-21 (2018); Scheufler, “Tissue Oxygenation and Capacity to Deliver O2 Do the Two Go Together,” Transfusion Apheresis Sci. 31:45-54 (2004)). Graft oxygenation is further impaired by the inevitable formation of a fibrotic capsule around the implant, which creates an additional barrier to oxygen transport (Avgoustiniatos et al., “Effect of External Oxygen Mass Transfer Resistances on Viability of Immunoisolated Tissue,” Ann. N. Y. Acad. Sci. 831:145 (1997)). Of all cell requirements, oxygen is at the lowest concentration with respect to its rate of consumption and is therefore often the most critically limited species in cell encapsulation (Tannock, “Oxygen Diffusion and the Distribution of Cellular Radiosensitivity in Tumours,” Br. J. Radiol. 45:515-24 (1972)).

Strategies to address inadequate oxygenation have included the induction of graft vascularization (Rouwkema et al., “Vascularization in Tissue Engineering,” Trends Biotechnol. 26:434-41 (2008)) device geometry and materials optimization (Ernst et al., Adv. Healthc. Mater. 8:1900423 (2019); Lewis, Doctor of Philosophy Thesis, Massachusetts Institute of Technology, (2008); Tomei et al., “Device Design and Materials Optimization of Conformal Coating for Islets of Langerhans,” Proc. Natl. Acad. Sci. U.S.A. 111:10514-9 (2014)) and exogenous oxygen supply. Oxygen delivery from an external source is often preferred as it can produce supraphysiological oxygen levels. For example, the decomposition of metal peroxides by hydrolysis has been shown to significantly improve graft oxygenation (Pedraza et al., “Preventing Hypoxia-induced Cell Death in Beta Cells and Islets Via Hydrolytically Activated, Oxygen-generating Biomaterials,” Proc. Natl. Acad. Sci. U.S.A. 109:4245 (2012); Gholipourmalekabadi, et al., “Oxygen-generating Biomaterials: a New, Viable Paradigm for Tissue Engineering?” Trends Biotechnol. 34:1010-1021 (2016)), however, with these technologies, the window of oxygen production is finite. Oxygen production by electrolysis has also been explored with success (Wu et al., “In Situ Electrochemical Oxygen Generation with an Immunoisolation Device,” Ann. N. Y. Acad. Sci. 875:105 (1999)) though this strategy is more complicated from an engineering perspective. Alternatively, the β-Air device (Beta-O2 Technologies) supports injectable oxygen into a gas-permeable chamber (Ludwig et al., “A Novel Device for Islet Transplantation Providing Immune Protection and Oxygen Supply,” Horm. Metab. Res. 42:918-22 (2010)). However, daily oxygen injections are required for graft survival. There is thus a need for developing a device which can support long-term high oxygenation of encapsulated cells, preferably without patient intervention.

The present application is directed to overcoming these and other deficiencies in the art.

### SUMMARY

The present application relates to a cell replacement device. The device includes a frame cap including a first connecting member and including one or more ports traversing a thickness of the frame cap. The device also includes a frame base including one or more walls defining an interior chamber, defining a first opening to the interior chamber on one side of the frame base and defining a second opening to the interior chamber on another side of the frame base, the first opening configured to receive the frame cap, the frame base further including a second connecting member constructed to connect with the first connecting member, wherein at least a portion of at least one of the one or more walls is porous. A mesh disposed adjacent the second opening is also included.

Another aspect of the present invention is directed to a device comprising a cell encapsulation module. The cell encapsulation module comprises a nanomembrane substrate; and a porous scaffold extending from the nanomembrane substrate.

Another aspect of the present disclosure is directed to a cell replacement device. The cell replacement device extends longitudinally from a first end to a second end and comprises the following features: (i) a cell encapsulation module, (ii) a frame cap proximal to the first end of the device, and (iii) a frame base proximal to the second end of the device. The cell encapsulation module of the device comprises a nanomembrane substrate and a porous scaffold extending from a surface of the nanomembrane substrate. The frame cap that is proximal to the first end of the device comprises a first surface and a second surface, said second surface proximal to the nanomembrane substrate of the cell encapsulation module, and one or more ports traversing the thickness of the frame cap through the first and second surfaces of the frame cap. The frame base that is proximal to the second end of the device comprises one or more walls defining an interior chamber, defining a first opening to the interior chamber on one side of the frame base, and defining a second opening to the interior chamber on another side of the frame base, the first opening configured to receive the frame cap, wherein at least a portion of at least one of the one or more walls is porous, and a mesh disposed adjacent to the second opening of the frame base. In accordance with this aspect of the disclosure, the frame base of the device is coupled to the frame cap of the device to form a housing that surrounds the cell encapsulation module of the device.

Another aspect of the present application relates to a method for delivering a therapeutic agent to a subject in need thereof. The method involves providing the cell replacement device of the present application and implanting the cell replacement device transcutaneously into a region of the subject suitable for delivering the therapeutic agent.

Another aspect of the present application relates to a cell encapsulation device kit. The kit includes a plurality of different cell replacement devices, each of the plurality of different cell replacement devices comprising a frame cap, a frame base, and a mesh. The frame cap includes a first connecting member and one or more ports traversing a thickness of the frame cap. The frame base includes one or more walls defining an interior chamber, defining a first opening to the interior chamber on one side of the frame base and defining a second chamber to the interior volume on another side of the frame base, the first opening configured to receive the frame cap. The frame base further includes a second connecting member constructed to connect with the first connecting member, wherein at least a portion of at least one of the one or more walls is porous, and wherein the mesh is disposed adjacent the second opening. A plurality of different cell encapsulation modules, each of the plurality of cell encapsulation modules being configured for insertion into the interior chamber of at least one of the plurality of different cell replacement devices is included, wherein each of the plurality of cell encapsulation modules including a nanomembrane substrate and a porous scaffold extending from the nanomembrane substrate.

Cell replacement therapy is emerging as a promising treatment platform for many endocrine disorders and hormone deficiency diseases. The survival of cells within delivery devices is, however, often limited due to low oxygen levels in common transplantation sites. Additionally, replacing implanted devices at the end of the graft lifetime is often infeasible and, if possible, generally requires invasive surgical procedures. Described herein is the design and testing of a modular transcutaneous biphasic cell delivery device which provides enhanced and unlimited oxygen supply by direct contact with the atmosphere. Critically, the cell delivery unit was demountable from the fixed components of the device, allowing for surgery-free refilling of the therapeutic cells. Mass transfer studies showed significantly improved performance of the biphasic device in comparison to subcutaneous controls. The device was also tested for islet encapsulation in an immunocompetent diabetes rodent model. Robust cell survival and diabetes correction was observed following a rat-to-mouse xenograft. Lastly, non-surgical cell refilling was demonstrated in dogs. These studies show the utility of this novel device for cell replacement therapies

### BRIEF DESCRIPTION OF THE DRAWINGS

FIGS. 1A-1J shows the design components and function of the biphasic device (“BP device”). FIG. 1A is an annotated schematic illustrating transcutaneous placement of the device in a subject. The fundamental components of the BP device include the frame cap (1), the ports that traverse the device (2), the cell encapsulation module (3), the frame base coupled to the frame cap (4). FIG. 1B shows digital images of an embodiment of an empty device for mouse implantation from the top view (left), side view (center), and bottom view (right). FIG. 1C is an annotated schematic illustrating device components and dimensions; schematic and digital image of the cell encapsulation module (left panel); schematic of each component, including the (top to bottom) titanium frame cap, cell encapsulation module, alginate-impregnated nylon mesh, and titanium frame base (center panel); schematic of the cell encapsulation module, featuring the PDMS sealing O-ring (right panel). FIG. 1D shows an SEM image of the PTFE nanomembrane. FIG. 1E shows a digital image of a water droplet on the PTFE nanomembrane. FIG. 1F shows a contact angle goniometer-captured image of a water droplet sliding on the PFC-infused PTFE nanomembrane after tilting to ˜5°. FIG. 1G shows SEM images of the PVDF-HFP scaffold displaying its spiral configuration and porous structure. FIG. 1H shows the chemical structure of the PVDF-HFP copolymer. FIG. 1I shows an SEM image of the nylon mesh. FIG. 1J shows a confocal image of the alginate-impregnated nylon mesh.

FIGS. 2A-2E show the cell replacement in Design 1 BP device in a dog. FIG. 2A shows a digital image of BP device illustrating dorsal transcutaneous placement. FIG. 2B shows a close-up digital image of BP device. FIGS. 2C-2D show the cell encapsulation module removed by unscrewing the frame cap. FIG. 2E shows the new frame cap (containing cell encapsulation module) screwed on to the frame base.

FIGS. 3A-3H show BP device testing and design evolution in rats. FIG. 3A show a schematic of Design 2 for rat transplantation, featuring elongated frame for implantation in the deeper subcutaneous space, a macroporous structure on the frame base, and extended handles for unscrewing the frame cap. FIG. 3B shows a digital image of the first design for the rat device. FIG. 3C shows a digital image of the device transplanted in the transcutaneous position immediately after surgery. FIG. 3D shows a schematic of Design 3, featuring a hexagon depression in the frame cap to enable screwing and unscrewing via a hex wrench. FIG. 3E shows digital images displaying the device fixed in the transcutaneous position 2 months post-surgery. FIG. 3F (Top) and FIG. 3G (side view) show digital images of the bottom face of one BP device within a fibrotic layer retrieved from a rat. FIG. 3H shows microscope images of Masson's trichrome stained slides at the device-host interface. Arrows indicate blood vessels and the asterisks (*) indicate the device side of the device-host interface.

FIGS. 4A-4B show the cell encapsulation module. FIG. 4A shows a digital image of the hydrogel- and (cell-free) cell encapsulation module showing the PTFE nanomembrane-attached PVDF-HFP spiral scaffold, and PDMS O-ring. FIG. 4B shows a digital image of the cell encapsulation module infused with alginate (islet media was mixed with the alginate solution prior to gelation providing a purple color for improved visualization).

FIGS. 5A-5J show diabetes correction in STZ-induced diabetic mice. FIG. 5A shows a schematic illustration of the BP device featuring a titanium frame for mouse implantation. FIG. 5B shows a schematic illustration of the islet encapsulation module.

FIG. 5C shows a microscope image of islets encapsulated within the spiral alginate hydrogel. FIG. 5D shows digital images showing the surgical procedure for fastening the device in the transcutaneous position: (left) a circular section of skin was excised and a purse-string suture pattern was placed in the surrounding skin; (center) the device was placed in the space of the excised skin; (right) the sutures were pulled tight and the device was fixed in the transcutaneous position. FIG. 5E shows blood glucose (BG) readings of mice receiving BP devices (n=5), subcutaneous transplantation controls (n=3), and nontreated diabetic mice (n=5); mean±SD; ***P<0.001 (BP device versus subcutaneous control), ***P<0.001 (BP device versus diabetic control). FIG. 5F shows IPGTT at day 7; n=5 for BP devices, n=3 for subcutaneous controls, n=5 for nontreated diabetic controls, n=5 for non-diabetic controls; mean±SD; ***P<0.001 (BP device versus subcutaneous control), ***P<0.001 (BP device versus diabetic control), n.s. (P>0.05; BP device versus non-diabetic control). FIG. 5G shows live/dead staining of islets from one retrieved BP device at day 15. FIG. 5H shows a static GSIS test of retrieved BP devices (n=3) and subcutaneous controls (n=3); mean±SEM; ***P<0.001 (2.8 mM versus 16.7 mM conditions for retrieved BP devices), n.s. (P>0.05; 2.8 mM versus 16.7 mM conditions for retrieved subcutaneous controls), ***P<0.001 (retrieved BP device versus retrieved subcutaneous control for both 2.8 mM and 16.7 mM conditions). FIG. 5I shows H&E staining of islets in one retrieved BP device at day 15. FIG. 5J shows immunohistochemical staining of islets in a retrieved BP device at day 15.

FIGS. 6A-6D show cell (MDA-MB-231) adhesion to the PFC-infused PTFE nanomembrane. FIG. 6A shows live/dead images of cells cultured on a cell culture dish (left) versus on a PFC-infused PTFE nanomembrane (right) show significantly impaired adhesion to the nanomembrane. FIG. 6B shows quantification of the number of cells found on surface of cell culture dish versus the PFC-infused PTFE nanomembrane. FIG. 6C shows SEM images, and FIG. 6D shows staining of F-actin and nuclei of cells exhibiting an elongated and extended morphology cultured on a cell culture dish (left) versus a spherical morphology cultured on the PFC-infused PTFE nanomembrane (right).

FIGS. 7A-7E show impaired bacterial adhesion and migration on the PFC-infused PTFE nanomembrane. Live/dead staining of S. aureus on (FIG. 7A) a control cover glass and (FIG. 7B) the PFC-infused PTFE nanomembrane reveals significantly reduced adhesion on the nanomembrane. FIG. 7C shows a schematic illustrating the experimental design of the bacterial migration study: S. aureus colonies grown on tryptic soy agar were inverted and placed on the top face of the PFC-infused nanomembrane. FIG. 7D shows live/dead staining of the bottom face of the PFC-infused PTFE nanomembrane revealed no migrated bacteria. FIG. 7E shows a digital image of tryptic soy agar plate incubated at 37° C. for 16 h following transfer/duplication from (left) the bacterial colonies and (right) the bottom face of the PFC-infused PTFE nanomembrane.

FIGS. 8A-8E show mass transfer. FIG. 8A shows simulation-predicted oxygen concentration profiles in the BP device, the BP device without the spiral scaffold, and a fully implanted subcutaneous control encapsulating islets. FIG. 8B shows quantification of spatially averaged islet oxygen concentration (islet number labelled #1 through #8 from left to right). FIG. 8C shows a surface plot of simulation-predicted oxygen profiles in the BP device and a subcutaneously transplanted control encapsulating dispersed (INS-1) cells. FIG. 8D shows simulation-predicted oxygen concentration in the BP device and a subcutaneously transplanted control along a horizontal cross section (labelled A-A) and a vertical cross section (labelled B-B) from surface plots shown in FIG. 8C. FIG. 8E shows a schematic of experimental design (top) and microscope images (bottom) of live/dead-stained INS-1 cells in BP device (left) exposed to the atmosphere while partially submerged in media at a pO₂ of 24 mmHg and a control alginate slab (right) fully submerged in media at a pO₂ of 24 mmHg.

FIG. 9A shows a top view of actual geometry of cell encapsulation module (left) and theoretical geometry (right) implemented in simulations. FIG. 9B is an annotated schematic of the components of the 2D islet encapsulation model and 2D axisymmetric INS-1 encapsulation model. FIG. 9C shows boundary conditions implemented in the computational model. A partial pressure of 160 mmHg or 24 mmHg at the top boundary was implemented to compare the difference in graft oxygenation between transcutaneous transplantation of the BP device versus the subcutaneous transplantation of control alginate slabs. All other boundary conditions were equivalent.

FIGS. 10A-10F show rat islet survival in control alginate slabs subcutaneously transplanted in the subcutaneous space of C57BL/6J mice. FIG. 10A shows a live/dead assay, FIG. 10B shows H&E staining, and FIG. 10C shows insulin/nucleus immunohistochemical staining of islets in an alginate slab before transplantation. FIG. 10D shows a live/dead assay, FIG. 10E shows H&E staining, and FIG. 10F shows insulin/nucleus immunohistochemical staining of retrieved islets after 15 days.

FIGS. 11A-11N show BP device transplantation and cell refilling in a dog. FIG. 11A are schematics illustrating the design evolution of the resin-based BP device; the first design (left) was upgraded to include a porous structure for tissue integration and was elongated to expose the cell encapsulation module to the deeper subcutaneous space; the second design (center) was upgraded to the final design (right) by including a hexagon depression for frame cap removal by a hex wrench and a trimmed rim diameter of the top of the frame base to reduce skin coverage. FIG. 11B is a digital image of the final BP device design. FIG. 11C shows a schematic and dimensions of the BP device components including a (top to bottom) PFC-infused PTFE nanomembrane, frame cap with a PDMS washer, cell encapsulation module, frame base with a porous exterior, anchor rings, and threading, and the alginate-impregnated nylon mesh. FIG. 11D is a digital image of the device in a dog at 1-month post-implantation. FIG. 11E is a digital image during the non-surgical refilling procedure: a hex wrench was placed in hexagon depression and twisted as the base was stabilized with forceps. FIG. 11F is a digital image of the device in a transcutaneous position after the frame cap (including the cell encapsulation module) has been removed. FIG. 11G is a digital image of the frame cap with the cell encapsulation module following retrieval at 1-month. FIG. 11H is a digital image of the BP device with the replaced cap containing encapsulated rat islets. FIG. 11I is a digital image of the BP device at 1-month post cell refilling. FIG. 11J is a digital image of the retrieved cell encapsulation module at 1-month post cell replacement. FIG. 11K shows H&E staining of rat islets from the retrieved BP device. FIG. 11L shows immunohistochemical staining of islets in the retrieved BP device. FIG. 11M is a schematic highlighting the porous structure on the exterior of the frame base. FIG. 11N shows H&E staining of the device and surrounding subcutaneous tissue showing tissue integration into the porous structure.

FIGS. 12A-12F are a summary of the BP device design evolution following iterative analysis in dogs. FIG. 12A shows Design 1, FIG. 12B shows Design 2, and FIG. 12C shows Design 3 device schematics and annotated longitudinal midline cross-sections. In cross-section images, blue indicates the Dental Resin and the brown indicates negative space (air). FIG. 12D shows the poor structural integrity of the handles for cap removal (left), infection of the cell encapsulation module (right), and poor device fixation observed after transplantation of Design 1 in a dog. FIG. 12E shows poor health observed after transplantation of Design 2 in a dog in tissue surrounding the device due coverage by top plate of frame base (left; arrow); poor structural integrity of handles observed during cap removal (right; arrows). FIG. 12F is a digital image of Design 3 (the final design) showing fit between hexagon depression in frame cap and hex wrench.

FIGS. 13A-13I show the implantation of Design 1 in a dog. FIG. 13A is a schematic of design 1. Digital images of the device from the (FIG. 13B) top and (FIG. 13C) bottom perspective are shown. FIGS. 13D-13H show digital images of surgical procedure for BP device Design 1 implantation. FIGS. 13D-13E show that a circular section of skin was excised; FIGS. 13F-13H show that the device was subsequently fastened by the anchor rings around the frame base using sutures (arrows indicate suture pathway and suture knot). FIG. 13I is a digital image of final position of the device immediately after surgery.

FIGS. 14A-14E show a C57BL/6J mouse BP device containing encapsulated rat islets presenting the concept of the macroporous structure for tissue fixation. FIG. 14A is a schematic of mouse BP device with porous structure integrated on the frame cap. FIG. 14B shows annotated digital images of the mouse BP device fabricated with Dental Resin (top view shown on top row; bottom view shown on bottom row). FIG. 14C shows a digital image of device transplanted in the transcutaneous position immediately after surgery. FIG. 14D is a digital image of device fixed in transcutaneous position without an observable adverse reaction after 1 month. FIG. 14E shows H&E-stained section of the cell encapsulation module showing robust islet health following retrieval after 1-month implantation.

FIGS. 15A-15B show fibrotic characterization of the BP device retrieved from mice. FIGS. 15A-15B are microscope images of Masson's trichrome stained slides at the device-host interface, including the underlying muscle layer. Arrows indicate blood vessels and the asterisks (*) indicate the device side of the device-host interface.

FIGS. 16A-16H show BP device testing and design evolution in rats. FIG. 16A is a schematic of Design 2 for rat transplantation, featuring elongated frame for implantation in the deeper subcutaneous space, a macroporous structure on the frame base, and extended handles for unscrewing the frame cap. FIG. 16B is a digital image of the first design for the rat device. FIG. 16C is a digital image of the device transplanted in the transcutaneous position immediately after surgery. FIG. 16D is a schematic of Design 3, featuring a hexagon depression in the frame cap to enable screwing and unscrewing via a hex wrench. FIG. 16E shows digital images displaying the device fixed in the transcutaneous position 2 months post-surgery. FIG. 16F show top and FIG. 16G show side view digital images of the bottom face of one BP device within a fibrotic layer retrieved from a rat. FIG. 16H shows microscope images of Masson's trichrome stained slides at the device-host interface. Arrows indicate blood vessels and the asterisks (*) indicate the device side of the device-host interface.

FIGS. 17A-17K show transplantation of BP device Design 2 in a dog. FIGS. 17A-17B show a schematic of Design 2, illustrating the porous structure on frame base for tissue fixation and anchor rings on the frame base for fastening the device in the transcutaneous position. FIG. 17C is a digital image of BP device Design 2. FIGS. 17D-17K show digital images showing surgical procedure for BP device Design 2 implantation. FIG. 17D shows that a circular incision was made in the epidermis; FIGS. 17E-17G show that subsequently, subcutaneous adipose tissue was excised; FIG. 17H shows that next, the device was placed in the space of the removed adipose tissue, and FIG. 17I shows that it was fastened via suture knots between the anchor rings and the surrounding subcutaneous tissue; FIG. 17J shows that next, a purse-string suture pattern was performed to close the incision. FIG. 17K is a digital image of final position of the device immediately after surgery.

FIGS. 18A-18C show the structural integrity of the alginate-impregnated nylon mesh and its attachment to the frame base. Digital images of (FIG. 18A) the bottom face of the BP device, the arrow indicating the alginate-impregnated alginate mesh attached to the frame base, (FIG. 18B) abrasion of the bottom face of the BP device onto a PDMS disk during testing, and (FIG. 18C) the bottom face of the BP device after testing, showing maintained structural integrity of the alginate-impregnated nylon mesh. Images were taken from Video S1.

FIGS. 19A-19C show several views of the biphasic cell replacement device Design 3. FIG. 19A provides a top view of the device (top panel) and bottom view of the device (bottom panel). FIG. 19B provides an exploded view of the device. FIG. 19C, top panel provides a cross-sectional view of the frame cap. FIG. 19C, middle panel provides an exploded view of the cell encapsulation module, and FIG. 19C, bottom panel provides a cross-sectional view of the frame base.

### DETAILED DESCRIPTION

The present application is directed to cell replacement devices and cell encapsulation modules for use in the cell replacement devices. The devices and modules described herein are useful for cell replacement therapies.

Accordingly, a first aspect of the present application is directed to a cell replacement device that comprises a frame cap including a first connecting member and including one or more ports traversing a thickness of the frame cap; a frame base including one or more walls defining an interior chamber, defining a first opening to the interior chamber on one side of the frame base, and defining a second opening to the interior chamber on another side of the frame base, the first opening configured to receive the frame cap. At least a portion of at least one of the one or more walls of the frame base of the device is porous. The frame base further includes a second connecting member constructed to connect with the first connecting member and a mesh disposed adjacent the second opening.

Another aspect of the present invention is directed to a device comprising a cell encapsulation module. In some embodiments, the device is the cell replacement device as described herein. However, it is appreciated that the cell encapsulation module is suitable for incorporation into other replacement therapy devices. In accordance with this aspect of the present disclosure, the cell encapsulation module comprises a nanomembrane substrate; and a porous scaffold extending from the nanomembrane substrate.

Another aspect of the present disclosure is directed to a cell replacement device as described herein comprising the cell encapsulation module as described herein. In accordance with this aspect of the present disclosure, the cell replacement device extends longitudinally from a first end to a second end, comprises the following features: (i) a cell encapsulation module, (ii) a frame cap proximal to the first end of the device, and (iii) a frame base proximal to the second end of the device. The cell encapsulation module of the device comprises a nanomembrane substrate and a porous scaffold extending from a surface of the nanomembrane substrate. The frame cap that is proximal to the first end of the device comprises a first surface and a second surface, said second surface proximal to the nanomembrane substrate of the cell encapsulation module, and one or more ports traversing the thickness of the frame cap through the first and second surfaces of the frame cap. The frame base that is proximal to the second end of the device comprises one or more walls defining an interior chamber, defining a first opening to the interior chamber on one side of the frame base, and defining a second opening to the interior chamber on another side of the frame base, the first opening configured to receive the frame cap. At least a portion of at least one of the one or more walls of the frame base of the device is porous. The frame base further comprises a mesh disposed adjacent to the second opening of the frame base. In accordance with this aspect of the disclosure, the frame base of the device is coupled to the frame cap of the device to form a housing that surrounds the cell encapsulation module of the device.

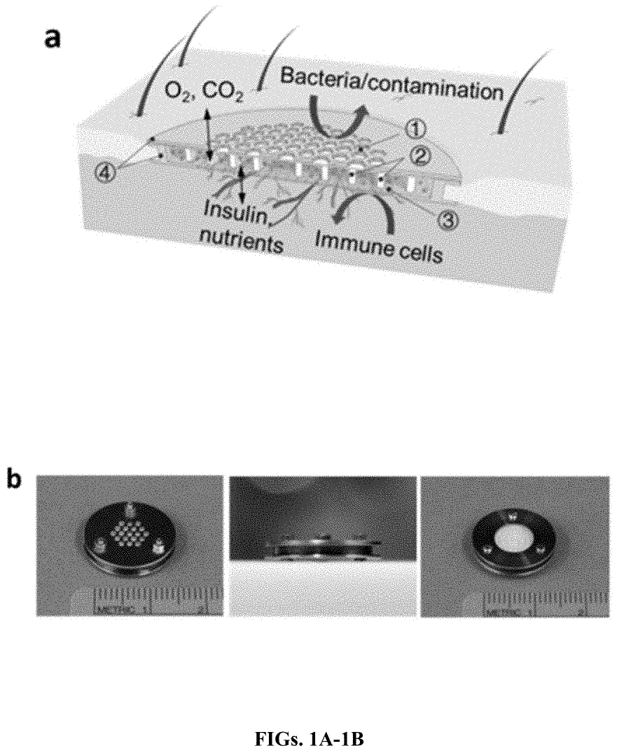

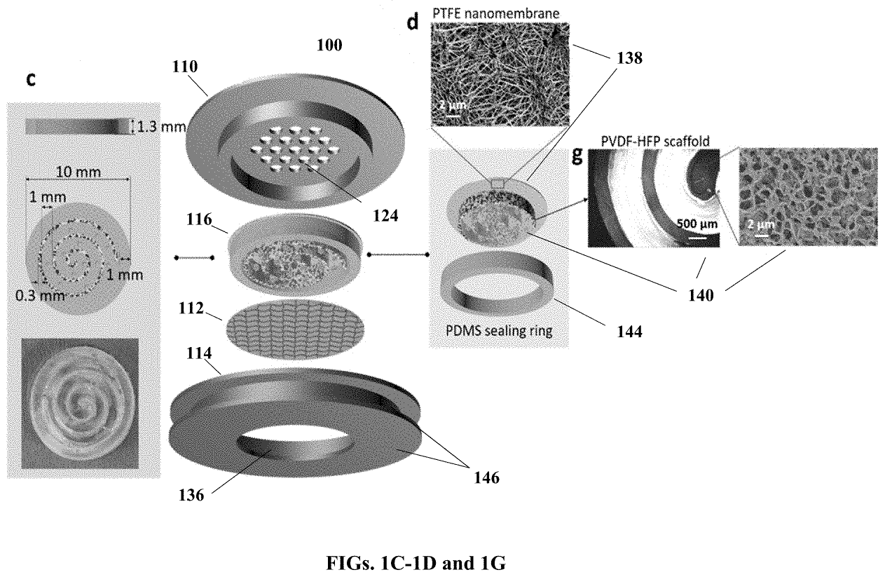

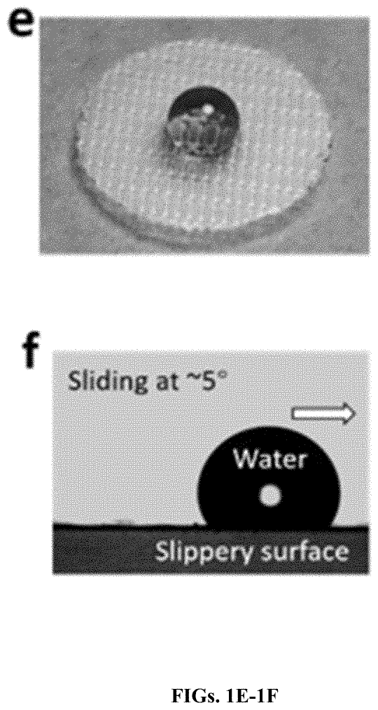

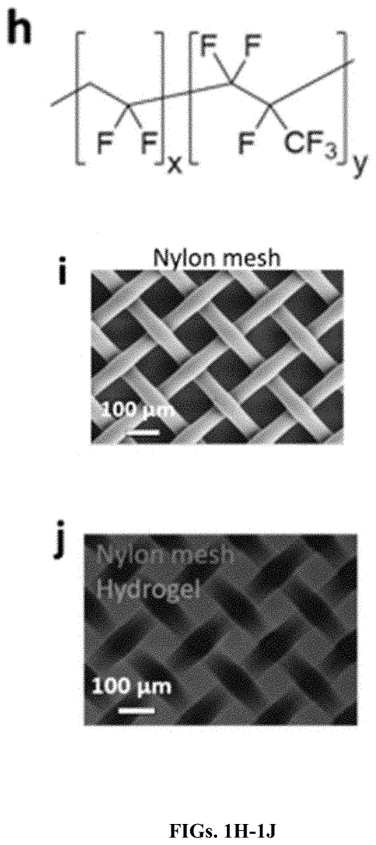

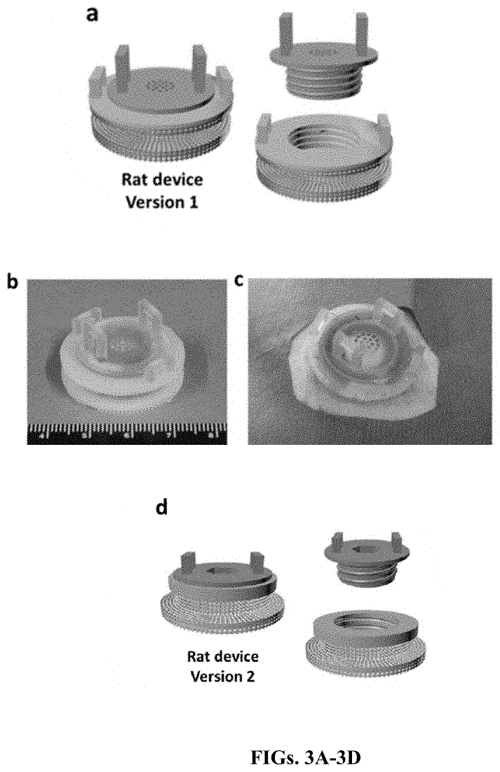

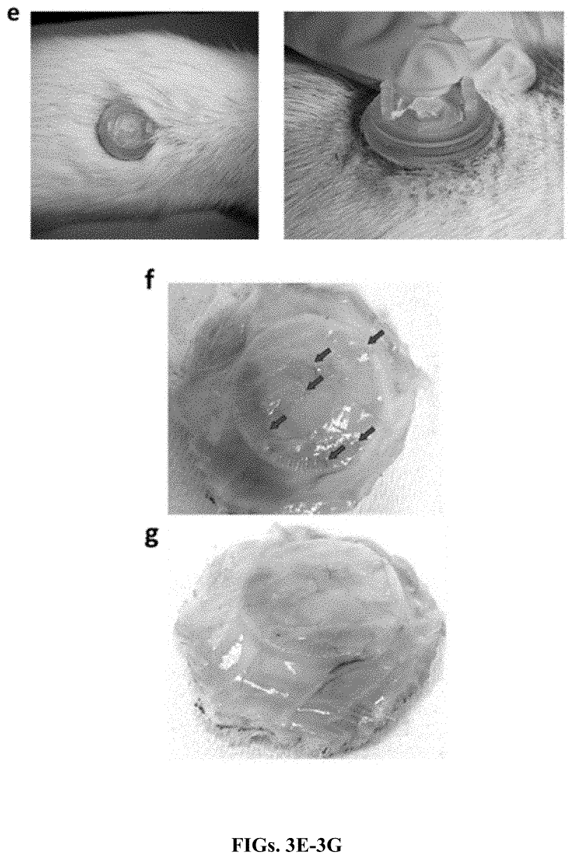

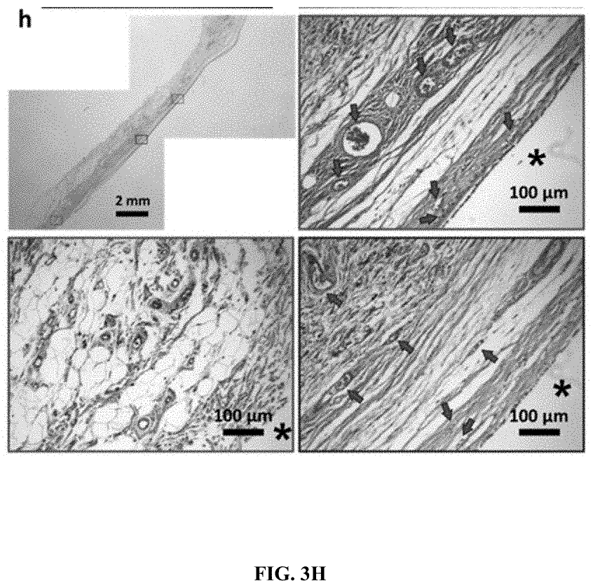

Exemplary embodiments of the cell replacement device will now be described herein with reference to Figures illustrating the various exemplary embodiments, see e.g, embodiments provided in FIGS. 1, 3, 11, 12, 14, 16, 17, 18 and 19. It will be appreciated that like structures/components of the device are provided with like reference designations.

In reference to FIG. 1C, exemplary embodiments of the cell replacement device 100 as described herein contain three primary components which include the frame cap 110, which is proximal to the first end of the device and comprises one or more ports 124 that traverse the thickness of the frame cap; frame base 114, which is proximal to the second end of the device and comprises one or more wall 146, a mesh 112, and a cell encapsulation module 116. The cell encapsulation module comprises a nanomembrane substrate 138, a porous scaffold 140, and a sealing ring 144.

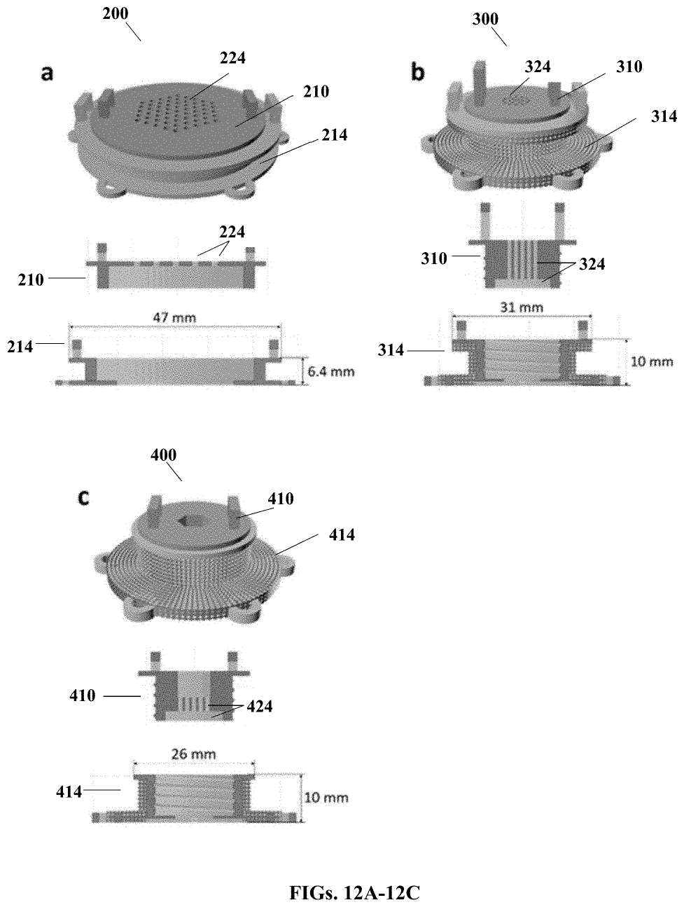

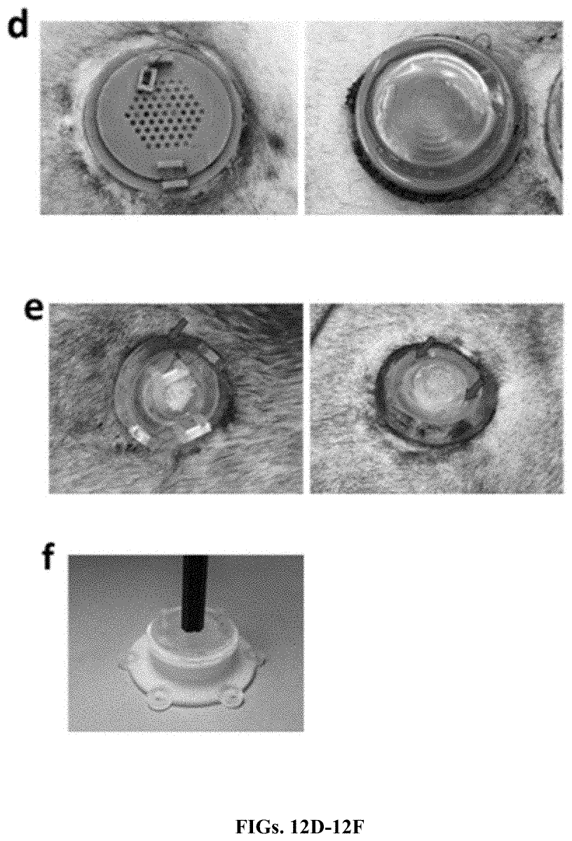

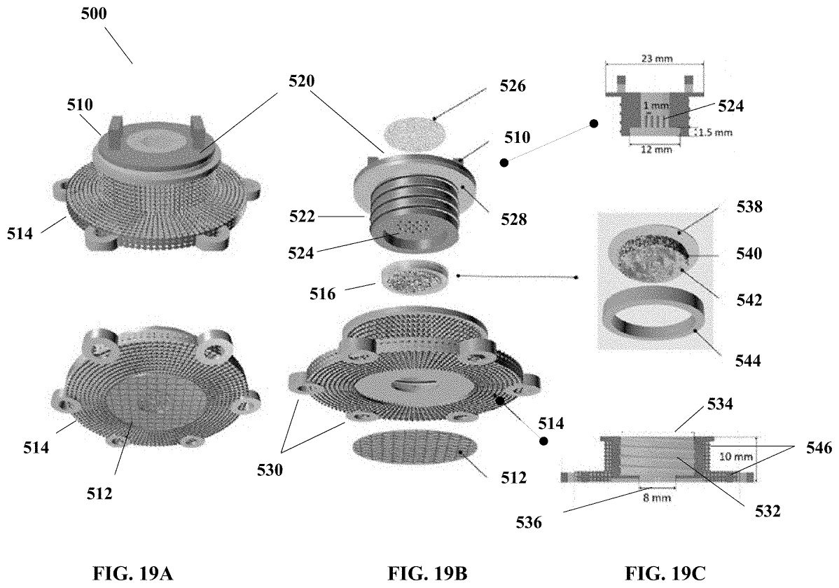

In reference to FIGS. 19A (top) and 19B, frame cap 510 is proximal to the first end of the device and comprises a first, exterior surface 520 and a second, interior surface 522. The second, interior surface 522 is proximal to the nanomembrane substrate 538 of the cell encapsulation module 516. The frame cap 510 further comprises one or more ports 524 that traverse the thickness of the frame cap through the first and second surfaces of the frame cap. In some embodiments, the one or more ports comprises a plurality of ports. As shown in FIGS. 12A-12C, the plurality of ports may individually traverse the thickness of the frame cap (see ports 224 of FIG. 12A) or merge into a single larger port on the exterior, interior (see ports 324 of FIG. 12B), or both surfaces (see ports 424 of FIG. 12C). The ports serve has a passageway for atmospheric gas to enter the device and feed the cells therein.

In some embodiments, second surface 522 of the frame cap 510 extends longitudinally toward the second end of the device. Embodiments of the device showing this longitudinal extension of the second surface of the frame cap are illustrated in the exploded view of FIG. 19B and cross-section vies of FIG. 19C (top panel). See also cross-sectional views of frame cap 310 and 410 provided in FIGS. 12B and 12C, respectively. The extent of the longitudinal extension is determined by desired implantation depth of the cell encapsulation module, e.g., shallow subcutaneous region vs. deep subcutaneous region.

In some embodiments, the frame cap comprises a coupling member suitable for coupling the frame cap to the frame base of the device. In some embodiments, and in reference to FIG. 19B, the coupling member is a threaded surface suitable for coupling the frame cap 510 to a threaded interior wall of the base frame 514.

In some embodiments, frame cap 510 is detachable from the device. In accordance with this embodiment, the first surface 520 of the frame cap 510 further comprises a means for removing or detaching the frame cap 510 from the device. In some embodiments, the frame cap comprises a small handle, tab, latch, screw, bolt, etc. that can be utilized to detach the frame cap from the frame base. In some embodiments, the frame cap comprises a depression or the like that fits a hex wrench or similar tool to facilitate cap removal from the remainder of the device or device opening to access the cell encapsulation module inside the device.

In reference to FIG. 19B, in some embodiments, frame cap 510 further comprises a porous nanomembrane 526 that serves as an additional barrier between the atmosphere-cell encapsulation interface. This porous nanomembrane 526 is a gas permeable material covering the ports on the first exterior surface of the device 520. The porous nanomembrane 526 allows the flow of atmospheric gas into the interior of the device through the ports 524, prevents dehydration of the cells inside of the device, and prevents contaminant entry into the device. Suitable frame cap nanomembrane film materials include any PFC-wettable nanomembrane film with a pore size below 0.2 microns. Examplary materials include, without limitation, polytetrafluoroethylene (PTFE), nylon, polycarbonate (PCTE), polyether ether ketone (PEEK), polyethersulfone (PES), polyester (PETE), polypropylene, polyvinylidene fluoride (PVDF). Inorganic nanomembrane films, such as aluminum oxide membranes, may also be suitable. In some embodiments, the frame cap nanomembrane material is an oil infused membrane. In some embodiments, the nanomembrane film is infused with perfluorinated carbon (PFC) oil. In some embodiments, the nanomembrane film is infused with silicon oil. In some embodiments, the nanomembrane film is infused with mineral oil.

In some embodiments, frame cap 510 further comprises a washer 528 that seals the frame cap 510 to the frame base 514 (see FIG. 19B). In some embodiments, the washer 528 is comprised of a silicon-based organic polymer material, such as polydimethylsiloxane (PDMS).

Frame base 514 of the device is proximal to the second end of the device. In reference to FIGS. 19B and 19C (bottom panel), frame base 514 comprises one or more walls that define an interior chamber 532 of the device into which the cell encapsulation module resides. The volume of this interior chamber may be from 0.1 mL to 100 mL. Walls of the frame base further define a first opening 534 leading into the interior chamber on one side of the frame base, and a second opening 536 leading into the interior chamber from the other side (exterior side) of the frame base. The first opening 534 of the frame base 514 is configured to receive the frame cap 510.

In some embodiments, at least a portion of at least one of the one or more walls of the frame base is macroporous. The porosity of the one or more walls should be about 500 microns to allow for tissue ingrowth and a strong, secure adhesion. In some embodiments, the walls defining the interior chamber of the frame base are macroporous. In some embodiments, all of the walls of the frame base are macroporous. In some embodiments, the frame base further comprises one or more anchor rings 530 around the periphery of the frame base 514 (FIGS. 19A, bottom panel and 19B). The anchor rings 530 are suitable for suturing the device to a biological substrate, e.g., tissue, bone, cartilage, etc. upon implantation into a subject.

The frame base 514 further comprises a mesh 512 that provides mechanical reinforcement at the device-host interface. In some embodiments, the mesh 512 is disposed of adjacent to the second opening 536 of the frame base 514 (see FIG. 19B). In some embodiments, the mesh 512 is disposed of adjacent to the first opening of the frame base 514 (see FIG. 1C). In some embodiments, the mesh 512 of the device comprises a nylon mesh (see FIG. 1I). In some embodiments, the nylon mesh is further coated with a hydrogel material (see FIG. 1J). A woven polyester mesh with thread diameter below 100 microns and a mesh opening larger than 100 microns is also suitable for use as a mesh. In some embodiments, the polyester mesh is further coated with a hydrogel material.

Suitable hydrogel materials include, without limitation, natural polymer hydrogel materials such as alginate, collagen, hyaluronate, fibrin, fibroin, agarose, chitosan, bacterial cellulose, elastin, keratin, gelatin, gelatin-methacryloyl, silk fibroin, glycosaminoglycans, dextran, agarose, matrigel, decellularized hydrogels, derivatives thereof and combinations thereof. In some embodiments, the natural polymer hydrogel material is a zwitterionically-modified natural hydrogel material, e.g., a zwitterionically-modified alginate material. Suitable zwitterionically modified alginates include, without limitation, those disclosed in Liu et al., “Zwitterionically Modified Alginates Mitigate Cellular Overgrowth for Cell Encapsulation,” Nat. Commun. 10(1):5262 (2019); and U.S. Patent Application Publication No. 20190389979 to Ma and Liu, which are hereby incorporated by reference in their entirety.

In some embodiments, the hydrogel material is a synthetic polymer hydrogel material, such as, polyethylene glycol, a polyethylene glycol derivative, poly(2-hydroxyethyl methacrylate), a poly(2-hydroxyethyl methacrylate) derivative, poly(acrylic acid), poly(ethylene oxide), poly(vinyl alcohol), polyphosphazene, poly(hydroxyethyl methacrylate), triazole-zwitterion hydrogels (TR-qCB, TR-CB, TR-SB), poly(sulfobetaine methacrylate), carboxybetaine methacrylate, poly[2-methacryloyloxyethyl phosphorylcholine, N-hydroxyethyl acrylamide, a copolymer thereof, a derivatives thereof, and a combination thereof.

In some embodiments, the frame cap and frame base of the cell replacement device are composed of a non-dissolvable, biocompatible material. In some embodiments, the frame cap and frame base are composed of the same non-dissolvable, biocompatible material. In some embodiments, the frame cap and frame base are composed of different non-dissolvable, biocompatible material. Suitable non-dissolvable, biocompatible material include medical grade metal, such as titanium, or a synthetic resin. In some embodiments, the non-dissolvable, biocompatible material is a polycaprolactone, poly(lactic acid) material. The frame cap and base can alternatively be composed of other biocompatible material that functions under physiologic conditions, including pH and temperature. Examples of suitable biocompatible materials include, but are not limited to, anisotropic materials, polysulfone (PSF), nano-fiber mats, polyimide, tetrafluoroethylene/polytetrafluoroethylene (PTFE; also known as Teflon®), ePTFE (expanded polytetrafluoroethylene), polyacrylonitrile, polyethersulfone, acrylic resin, cellulose acetate, cellulose nitrate, polyamide, as well as hydroxylpropyl methyl cellulose (HPMC).

Residing within the interior chamber 532 of the frame base 514 of the cell replacement device is the cell encapsulation module 516 (FIG. 19B). Exemplary components of the cell encapsulation module as shown in FIG. 19C (middle panel) include the nanomembrane substrate 538, porous scaffold 540, hydrogel 542, and sealing ring 544.

In some embodiments, the cell encapsulation module comprises a porous nanomembrane substrate 538 that is gas permeable and resistant to bacterial infiltration and growth. In some embodiments, the nanomembrane substrate 538 of the cell encapsulation module 516 comprises a polytetrafluoroethylene (PTFE) nanomembrane, nylon, polycarbonate (PCTE), polyether ether ketone (PEEK), polyethersulfone (PES), polyester (PETE), polypropylene, or polyvinylidene fluoride (PVDF). Inorganic nanomembrane films, such as aluminum oxide membranes, may also be suitable. In some embodiments, the nanomembrane substrate 538 comprises a non-fluorinated polymer chemically modified with fluoroalkysilanes. In some embodiments, the nanomembrane substrate 538 of the cell encapsulation module 516 is infused with an oil, such as a perfluorinated carbon oil, silicon oil, or mineral oil.

The nanomembrane substrate 538 of the cell encapsulation module 516 is a porous, gas permeable material that allows passage of atmospheric gas, but prevents the infiltration of bacteria and other contaminants. In some embodiments, the nanomembrane substrate has a nanoporosity of between 50 and 500 nm.

In reference to FIG. 19C (middle panel), the cell encapsulation module 516 further comprises a porous scaffold 540 extending from a surface of the nanomembrane substrate 538 that functions to support cell viability. In some embodiments, the porous scaffold 540 of the cell encapsulation module comprises a fluorinated polymer material. In some embodiments, the fluorinated polymer material is poly(vinylidene fluoride-co-hexafluoropropylene) (PVDF-HFP). In some embodiments, the porous scaffold of the cell encapsulation module comprises a non-fluorinated polymer material chemically modified with fluoroalkysilanes.

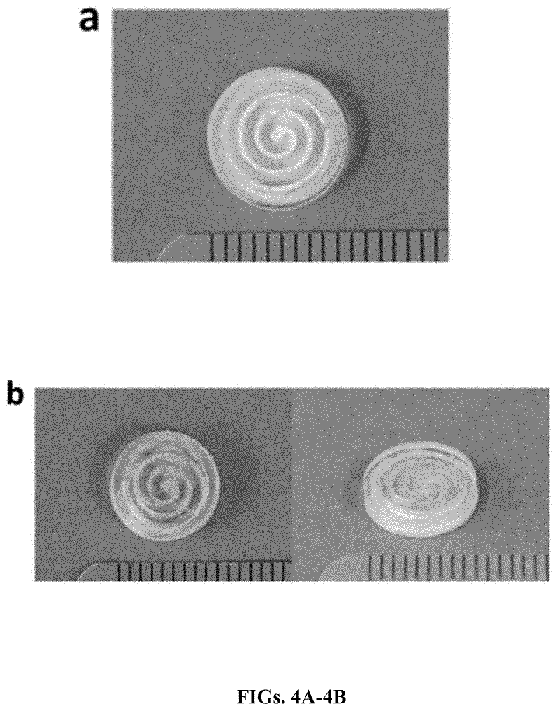

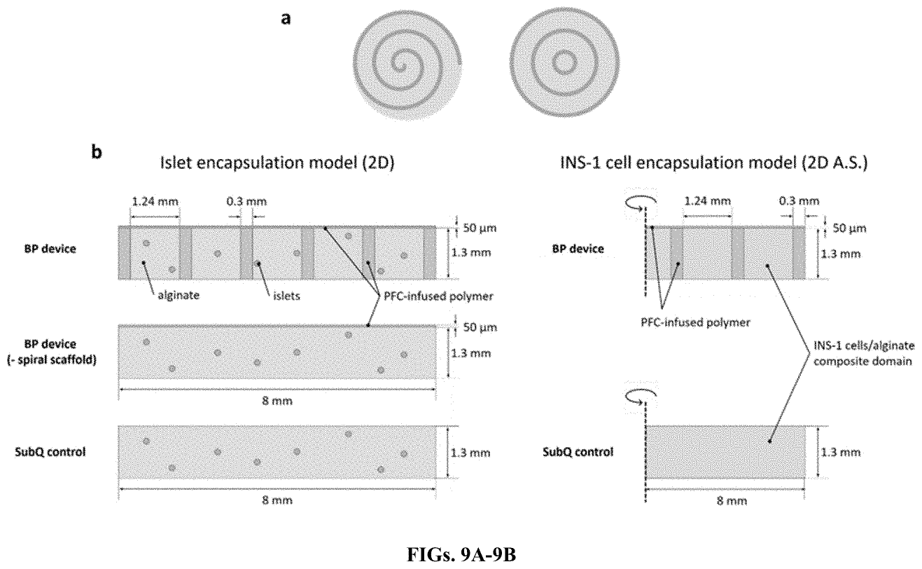

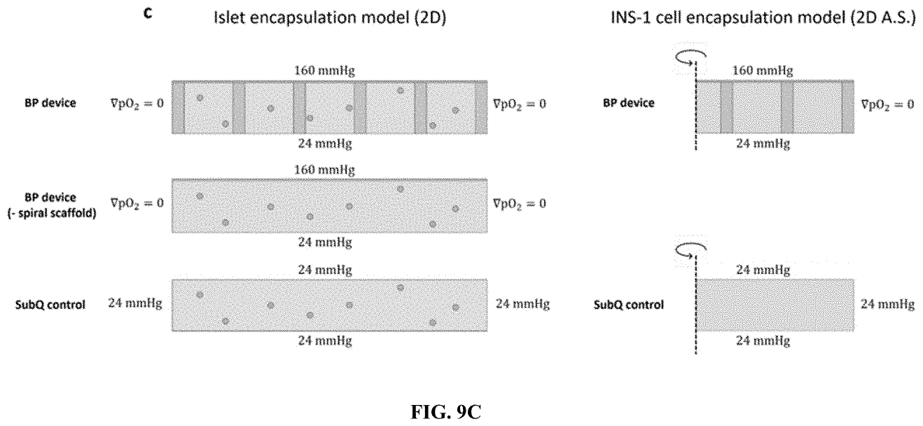

The porous scaffold can assume any geometry or design that facilitates cell growth and/or cell survival within the cell encapsulation module of the cell replacement device. As described herein, in some embodiments, the porous scaffold is a three dimensional spiral configuration as shown in FIG. 1C (see also FIGS. 4A-4B and 9A-9C). In reference to FIG. 19C (middle panel), the porous scaffold 540 is surrounded and/or encapsulated by a hydrogel material 542 that is suitable for supporting cell growth and survival. Suitable hydrogel materials include, without limitation natural polymer hydrogel material and synthetic polymer hydrogel material. Exemplary natural polymer hydrogel materials include, without limitation, alginate, collagen, hyaluronate, fibrin, fibroin, agarose, chitosan, bacterial cellulose, elastin, keratin, and combinations thereof. In some embodiments, the natural polymer hydrogel material is a zwitterionically-modified natural polymer hydrogel material. Suitable zwitterionically modified alginates include, without limitation, those disclosed in Liu et al., “Zwitterionically Modified Alginates Mitigate Cellular Overgrowth for Cell Encapsulation,” Nat. Commun. 10(1):5262 (2019); and U.S. Patent Application Publication No. 20190389979 to Ma and Liu, which are hereby incorporated by reference in their entirety.

In some embodiments, the hydrogel material is a synthetic polymer hydrogel material, such as, polyethylene glycol, a polyethylene glycol derivative, poly(2-hydroxyethyl methacrylate), a poly(2-hydroxyethyl methacrylate) derivative, poly(acrylic acid), poly(ethylene oxide), poly(vinyl alcohol), polyphosphazene, poly(hydroxyethyl methacrylate), triazole-zwitterion hydrogels (TR-qCB, TR-CB, TR-SB), poly(sulfobetaine methacrylate), carboxybetaine methacrylate, poly[2-methacryloyloxyethyl phosphorylcholine, N-hydroxyethyl acrylamide, a copolymer thereof, a derivatives thereof, and a combination thereof.

In some embodiments, the porous scaffold and/or the hydrogel material surrounding the porous scaffold of the cell encapsulation module, comprise one or more biologically active agents. Suitable biologically active agents include, without limitation, a protein, peptide, antibody or antibody fragment thereof, antibody mimetic, a nucleic acid, a small molecule, a hormone, a growth factor, an angiogenic factor, a cytokine, an anti-inflammatory agent, and combinations thereof.

In some embodiments, the porous scaffold and/or the hydrogel material surrounding the porous scaffold of the cell encapsulation module comprise one or more cell factors to enhance cell growth, differentiation, and/or survival. Suitable cell factors include, without limitation, glutamine, non-essential amino acids, epidermal growth factors, fibroblast growth factors, transforming growth factor/bone morphogenetic proteins, platelet derived growth factors, insulin growth factors, cytokines, fibronectin, laminin, heparin, collagen, glycosaminoglycan, proteoglycan, elastin, chitin derivatives, fibrin, and fibrinogen, FGF, bFGF, acid FGF (aFGF), FGF-2, FGF-4, EGF, PDGF, TGF-beta, angiopoietin-1, angiopoietin-2, placental growth factor (P1GF), VEGF, PMA (phorbol 12-myristate 13-acetate), combinations thereof.

The various embodiments of the cell replacements devices described and shown herein are in not intended to be limited to certain device size, shape, design, volume capacity, and/or materials used to make the cell replacement devices, so long as one or more of the above components are achieved.

The cell replacement device may further comprise a preparation of cells suspended in the hydrogel material within the cell encapsulation module. Encapsulation provides a protective barrier that hinders elements of the host immune system from destroying the cells. This allows the use of unmatched human or even animal tissue, without immunosuppression of the recipient and therefore results in an increase in the diversity of cell types that can be employed in therapy. Additionally, because the implanted cells are retained in the device, their encapsulation prevents the inherent risk of tumor formation otherwise present in some cell-based treatments.

In some embodiments, the preparation of cells is a preparation of single cells or a preparation of cell aggregates. In some embodiments, the preparation of cells is a preparation of primary cells or a preparation of immortalized cells. In some embodiments, the preparation of cells is a preparation of mammalian cells. In some embodiments, the preparation of cells is selected from the group consisting of a preparation of primate cells, rodent cells, canine cells, feline cells, equine cells, bovine cells, and porcine cells. In some embodiments, the preparation of cells is a preparation of human cells. In some embodiments, the preparation of cells is a preparation of stem cells or stem cell derived cells. In some embodiments, the stem cells are pluripotent, multipotent, oligopotent, or unipotent stem cells. In some embodiments, the preparation of stem cells is selected from the group consisting of embryonic stem cells, epiblast cells, primitive ectoderm cells, primordial germ cells, and induced pluripotent stem cells. In some embodiments, the preparation of cells is a preparation of cells selected from the group consisting of smooth muscle cells, cardiac myocytes, platelets, epithelial cells, endothelial cells, urothelial cells, fibroblasts, embryonic fibroblasts, myoblasts, chondrocytes, chondroblasts, osteoblasts, osteoclasts, keratinocytes, hepatocytes, bile duct cells, islet cells, thyroid, parathyroid, adrenal, hypothalamic, pituitary, ovarian, testicular, salivary gland cells, adipocytes, embryonic stem cells, mesenchymal stem cells, neural cells, endothelial progenitor cells, hematopoietic cells, precursor cells, mesenchymal stromal cells, Baby Hamster Kidney (BHK) cells, Chinese Hamster Ovary cells, Human Amniotic Epithelial (HAE) cells, choroid plexus cells, chromaffin cells, adrenal chromaffin cells, pheochomocytoma cell line PC12, human retinal pigment epithelium cells, recombinant human retinal pigment epithelium cells, NGF-secreting Baby Hamster Kidney (BHK) cells, human bone marrow-derived stem cells transfected with GLP-1, BDNF-producing fibroblasts, NGF-producing cells, CNTF-producing cells, BDNF-secreting Schwann cells, IL-2-secreting myoblasts, endostatin-secreting cells, and cytochrome P450 enzyme overexpressed feline kidney epithelial cells, myogenic cells, embryonic stem cell-derived neural progenitor cells, irradiated tumor cells, proximal tubule cells, neural precursor cells, astrocytes, genetically engineered cells, e.g., a preparation of cells engineered to recombinantly express a therapeutic agent.

In some embodiments, the preparation of cells is a preparation of any one or more of endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof.

In some embodiments, the preparation of cells in the cell encapsulation module comprises a cell density of between 1×10³ to 1×10¹⁰ cells/mL. In some embodiments, the preparation of cells in the cell encapsulation module comprises a cell density of between 1×10⁴ to 1×10⁹ cells/mL. In some embodiments, the preparation of cells in the cell encapsulation module comprises a cell density of between 1×10⁵ to 1×10⁸ cells/mL. In some embodiments, the preparation of cells in the cell encapsulation module comprises a cell density of between 1×10⁶ to 1×10⁷ cells/mL.

Another aspect of the disclosure relates to a method for delivering a therapeutic agent to a subject in need thereof. This method involves providing the cell replacement device as described herein and implanting the device transcutaneously into a region of the subject suitable for delivering the therapeutic agent.

In some embodiments, the subject in need of treatment thereof, is a subject having diabetes, and the method of delivering a therapeutic agent to the subject involves implanting the cell replacement device into the subject having diabetes. In accordance with this embodiment, the one or more therapeutic agents of the cell replacement device is insulin, glucagon, or a combination thereof. In some embodiments, the insulin, glucagon, or combination thereof is released from a preparation of cells positioned in the cell encapsulation module of the cell replacement device. In some embodiments, the preparation of cells comprises a preparation of islets. In some embodiments, the preparation of islets is a preparation of primate islets, rodent islets, canine islets, feline islets, equine islets, bovine islets, or porcine islets. In some embodiments, the preparation of islets is derived from a preparation of stem cells. In some embodiments, the preparation of stem cells is a preparation of pluripotent, multipotent, oligopotent, or unipotent stem cells. In some embodiments, the preparation of stem cells is a preparation comprising embryonic stem cells, epiblast cells, primitive ectoderm cells, primordial germ cells, and induced pluripotent stem cells. In some embodiments, the preparation of cells comprises an islet density between 1×10³ to 6×10⁵ islet equivalents (IEQs)/mL.

In some embodiments, the subject in need of treatment thereof is a subject having a bleeding disorder, and the method of delivering a therapeutic agent to the subject involves implanting the cell replacement device as described herein into the subject having the bleeding disorder. In accordance with this embodiment, the bleeding disorder can be any bleeding disorder, such as hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency. In some embodiments, the one or more therapeutic agents is a blood clotting factor released from a preparation of cells positioned in the cell encapsulation module of the cell replacement device. In some embodiments, the preparation of cells comprises recombinant myoblasts, mesenchymal stromal cells, induced pluripotent stem cell derived endothelial cells, or a combination thereof. In some embodiments, the blood clotting factor is selected from the group consisting of Factor I, Factor II, Factor V, Factor VII, Factor VIII, Factor IX, Factor X, Factor XI, Factor XII, Factor XIII, and combinations thereof.

In some embodiments, the subject in need of treatment thereof is a subject having a lysosomal storage disorder, and the method of delivering a therapeutic agent to the subject involves implanting the cell replacement device as described herein into the subject having the lysosomal storage disorder. In some embodiments, the one or more therapeutic agents is an enzyme released from a preparation of cells positioned in the cell encapsulation module of the cell replacement device. In some embodiments, the preparation of cells comprises hematopoietic stem cells, fibroblasts, myoblasts, Baby Hamster Kidney (BHK) cells, Chinese Hamster Ovary cells, Human Amniotic Epithelial (HAE) cells, or combinations thereof. In some embodiments, the enzyme is selected from the group consisting of α-L-iduronidase, Iduronate-2-sulfatase, α-glucuronidase, Arylsulfatase A, alpha-Galactosidase A, and combinations thereof.

In some embodiments, the subject in need of treatment thereof is a subject having cancer, and the method of delivering a therapeutic agent to the subject involves implanting the cell replacement device as described herein into the subject having cancer disorder. In some embodiments, the one or more therapeutic agents is a therapeutic molecule released from a preparation of cells in the cell encapsulation module of the cell replacement device. In some embodiments, the preparation of cells comprises IL-2-secreting myoblasts, endostatin-secreting cells, Chinese Hamster Ovary cells, and cytochrome P450 enzyme overexpressed feline kidney epithelial cells. In some embodiments, the therapeutic molecule is selected from IL-2, endostatin, cytochrome P450 enzyme, and combinations thereof.

In some embodiments, the subject in need of treatment thereof is a subject having kidney failure and the method of delivering a therapeutic agent to the subject involves implanting the cell replacement device as described herein into the subject having kidney failure. In some embodiments, the one or more therapeutic agents is a therapeutic molecule released from a preparation of cells positioned in the cell encapsulation module of the cell replacement device. In some embodiments, the preparation of cells comprises renal proximal tubule cells, mesenchymal stem cells, and a combination thereof.

In some embodiments, the subject in need of treatment thereof is a subject having chronic pain and the method of delivering a therapeutic agent to the subject involves implanting a cell replacement device as described herein into the subject having chronic pain. In some embodiments, chronic pain is chronic pain caused by degenerative back and knee, neuropathic back and knee, or cancer. In some embodiments, the one or more therapeutic agents is a therapeutic molecule released from a preparation of cells positioned in the cell encapsulation module of the cell replacement device. In some embodiments, the preparation of cells comprises chromaffin cells, neural precursor cells, mesenchymal stem cells, astrocytes, and genetically engineered cells, or a combination thereof. In some embodiments, the therapeutic molecule is selected from the group consisting of catecholamine, opioid peptides, enkephalins, and combinations thereof.

In accordance with all of the methods described herein a “subject” refers to any animal. In some embodiments, the subject is a mammal. Exemplary mammalian subjects include, without limitation, humans, non-human primates, dogs, cats, rodents (e.g., mouse, rat, guinea pig), horses, cattle and cows, sheep, and pigs. In some embodiments, the subject is a human.

In accordance with the methods of treatment described herein, it may be necessary or desirable to replace or replenish the cells of the cell replacement device periodically. The cell replacement device described herein facilitates non-surgical cell replacement or replenishment. To replace cells of an implanted cell replacement device, the frame cap is removed from the device, and a fresh preparation of cells or new cell encapsulation module is added to the device. The frame cap is then replaced on the device.

Another aspect of the present disclosure is directed to a cell encapsulation device kit. This cell encapsulation device kit comprises a plurality of different cell replacement devices, each of the plurality of different cell replacement devices comprising a frame cap, a frame base, and a mesh. The frame cap includes a first connecting member and one or more ports traversing a thickness of the frame cap. The frame base includes one or more walls defining an interior chamber, defining a first opening to the interior chamber on one side of the frame base and defining a second opening to the interior chamber on another side of the frame base. The first opening of the frame base is configured to receive the frame cap, wherein the frame base further includes a second connecting member constructed to connect with the first connecting member. At least a portion of at least one of the one or more walls of the frame base is porous. The mesh of the frame base is disposed adjacent the second opening. The kit further includes a plurality of different cell encapsulation modules, each of the plurality of cell encapsulation modules being configured for insertion into the interior chamber of at least one of the plurality of different cell replacement devices, wherein each of the plurality of cell encapsulation modules including a nanomembrane substrate and a porous scaffold extending from the nanomembrane substrate.

### EXAMPLES

The examples below are intended to exemplify the practice of embodiments of the disclosure but are by no means intended to limit the scope thereof.

### Materials and Methods

Animals. C57BL/6J mice for transplantation experiments were purchased from the Jackson Laboratory (Bar Harbor, Me.). Sprague-Dawley rats for isolation of pancreatic islets were obtained from Charles River Laboratories (Wilmington, Mass.). Beagle dogs were obtained from Marshall Bioresources (Clyde, N.Y.). All animal procedures were approved by the Cornell Institutional Animal Care and Use Committee.

Characterizations. Scanning electron microscopy (SEM) was performed by using a field emission scanning electron micro-analyzer (LEO 1550). Contact angles were measured using a contact angle goniometer (Rame-Hart 500). H&E staining images were taken using an Aperio Scanscope (CS2). Optical and fluorescent microscopic images were taken using a digital inverted microscope (EVOS fl). Confocal images were taken by a Laser Scanning Confocal Microscope (LSM 710).

Preparation of the porous PVDF-HFP scaffold on the PTFE nanomembrane. PVDF-HFP (PVDF-HFP, Mw ˜455,000 Da, Sigma-Aldrich) was dissolved in acetone at a concentration of 15% (w/v). The solution was filled into a 3D printed spiral polylactic acid (PLA) mold, under which was the PTFE nanomembrane (Laminated, pore size 0.2 μm, Sterlitech). Porous PVDF-HFP was built upon and attached to the PTFE nanomembrane via a phase separation process in water/alcohol (v/v=1/1) bath. After a solidification process in a water bath, the mold with the PVDF-HFP and PTFE nanomembrane were immersed in ethanol and hexane, followed by air drying at ambient temperature. Then, the PVDF-HFP scaffold, attached to the PTFE nanomembrane, was obtained by extracting the PLA mold with chloroform.

Bacterial adhesion test. A single colony of Staphylococcus aureus ATCC 3359 was transferred to 5 mL of tryptic soy broth and incubated at 37° C. for 16 h. The cultured bacterial suspension was diluted to the concentration of 10⁶ cells mL⁻¹. The diluted bacterial suspension was added into a 24 well plate (1 mL well⁻¹); subsequently, 12 mm diameter PFC-infused PTFE nanomembranes and control cover glass samples were immersed in the bacterial suspension. After incubation at 37° C. for 6 h, the samples were taken out from the culture medium and rinsed twice with PBS buffer. The samples were stained with a live/dead BacLight bacterial viability kit (Invitrogen) and observed via fluorescence microscopy (Fluorescent Cell Imager, ZOE).

Bacterial passage test. Staphylococcus aureus ATCC 3359 was cultured on a tryptic soy agar plate (Hardy Diagnostics) at 37° C. for 16 hours. Pieces of tryptic soy agar with bacterial colonies were cut from the cultured tryptic soy agar plate using a 6 mm biopsy punch. The pieces of tryptic soy agar were transferred on 47 mm diameter PFC-infused PTFE nanomembranes (i.e. the face with bacterial colonies was inverted and placed in contact with the nanomembrane) and incubated at 37° C. for 6 hours. The bottom face of PFC-infused PTFE nanomembrane was stained with a live/dead BacLight bacterial viability kit and observed via fluorescence microscopy. Additionally, the bottom face of the membrane was swabbed and transferred/duplicated on a new tryptic soy agar plate and incubated for at 37° C. for 16 hours (bacterial colonies were swabbed and transferred/duplicated on the same tryptic soy agar plate as a positive control).

Fabrication of the metal biphasic (BP) device for mice. The metal BP device frames were fabricated using titanium at the Cornell Computer Numerical Control (CNC) machine shop. Device components were autoclaved for sterilization. The PTFE membrane and PVDF-HFP scaffold were lubricated with PFC oil (Krytox® GPL103, DuPont) and placed into the frame cap by the inclusion of a PDMS O-ring. Cells were pre-mixed with sterile 2% (w/v) alginate solution (Pronova SLG100, FMC BioPolymer) and filled around the PVDF-HFP scaffold via pipet. The components were assembled according to the scheme shown in FIG. 1C and the device frames were tightened by three pairs of screws and nuts. The assembled device was soaked in 95 mM CaCl₂) and 5 mM BaCl₂ buffer for 5 minutes to crosslink the alginate within the cell encapsulation module. Lastly, all devices which contained encapsulated cells were washed with 0.9% NaCl and transferred into cell culture medium.

Fabrication of the resin biphasic device for mice, rats, and dogs. The 3D models of device frames were designed using Autodesk 3ds Max software and saved as a stereolithography (.stl) file allowing direct import into the printer software. The mouse device only had one frame, whereas the rat and dog devices comprised two parts—the frame cap and the frame base. The device frames were printed using a 3D printer (Form2, Formlabs) with the Class IIa biocompatible Dental LT resin.

To fabricate a mouse BP device, the device frame, PFC oil (Krytox® GPL103, DuPont) lubricated PTFE nanomembrane and PVDF-HFP scaffold, PDMS O-ring, and nylon mesh (diameter of 12 mm, pore size of 100 Component Supply) were autoclaved for sterilization. The components were assembled according to the scheme shown in FIG. 12B, and the PTFE membrane and PVDF-HFP scaffold were placed into the frame cap by the inclusion of a PDMS O-ring. Cells were pre-mixed with sterile 2% (wt/vol) alginate solution (SLG100, FMC BioPolymer) and filled around the PVDF-HFP scaffold via pipet. The nylon mesh was placed at the bottom of device, and a thin layer of alginate was applied to cover the nylon mesh. The assembled device was soaked in 95 mM CaCl₂) and 5 mM BaCl₂ buffer for 5 minutes to crosslink the alginate in cell module and mesh. Lastly, the device was washed with 0.9% NaCl solution and transferred into cell culture medium. After device implantation, an additional PTFE nanomembrane was attached on the top of device using a super glue (3M) and a drop of PFC oil was applied, which spontaneously infiltrated and impregnated the membrane.

To fabricate BP devices for rat and dog transplantation, the device frames, PFC oil-lubricated PTFE nanomembrane and PVDF-HFP scaffold, PDMS O-ring, PDMS washer, and nylon mesh were autoclaved for sterilization. The components were assembled according to the scheme shown in FIG. 12C, a PDMS washer was attached below the edge of frame cap, and the PTFE membrane and PVDF-HFP scaffold were placed into the frame cap by the inclusion of a PDMS O-ring. Then, the device frames were tightened by screwing the external/male thread of the frame cap into the internal/female thread of the frame base. Cells were pre-mixed with sterile 2% (w/v) alginate solution (SLG100, FMC BioPolymer) and filled around the PVDF-HFP scaffold via pipet from the bottom of the device. The device was soaked in 95 mM CaCl₂) and 5 mM BaCl₂ buffer for 5 minutes to crosslink the alginate within the cell encapsulation module. Then, an alginate-coated nylon mesh was attached to the bottom of the device. Lastly, the device was washed with 0.9% NaCl solution and transferred into cell culture medium. After implantation, an additional PTFE nanomembrane was attached on the top of device using a super glue (3M) and a drop of PFC oil was applied, which spontaneously infiltrated and impregnated the membrane.

In vitro cell attachment and cell viability on PFC-infused PTFE nanomembrane. MDA-MB-231 cells were cultured in DMEM medium (Gibco) supplemented with 2 mM glutamine (Gibco), 10% (v/v) heat inactivated fetal bovine serum (Gibco), and 1% penicillin/streptomycin (Gibco). MDA-MB-231 cells were seeded in a 12-well plate (2×10⁵ cells per well) with or without 22 mm diameter PFC-infused PTFE nanomembranes at the bottom of plate. After 24 hours of culture at 37° C. and 5% CO₂, the membranes and control wells were briefly rinsed with PBS to remove any unattached cells. The attached cells on the PFC-infused PTFE nanomembranes and control wells were stained with calcein AM and ethidium homodimer-1 (ThermoFisher) to evaluate the cell attachment and cell viability. The morphology of cells on PFC-infused PTFE nanomembranes and control wells were examined by scanning electron microscopy (SEM, samples were fixed in 4% paraformaldehyde and serially dehydrated in increasing concentrations of ethanol) and f-actin immunohistochemical staining (Abcam).

Mass Transfer Modeling. A computational framework was developed to simulate oxygen transport in the BP device and subcutaneously implanted controls (FIG. 9). Two models were developed: (1) a cell cluster (islet) encapsulation model (FIG. 9B), and (2) a dispersed-cell (INS-1) encapsulation model (FIG. 9C). To reduce computational complexity and time, the spiral scaffold structure was assumed to be sufficiently similar to concentric cylinders such that analysis could be performed in 2D or 2D axisymmetric geometries (FIG. 9A). For both models, oxygen transport was assumed to be at a steady state and governed by the diffusion-reaction mass balance equation with negligible convection (Equation 1).

D_{O}_{₂}_{,i}∇²c_{O}_{₂}=R_{O}_{₂} (1)

Here, D_{O}_{₂}_{,i} represents the diffusivity of oxygen in domain i, ∇² represents the Laplacian

$$( i . e . \frac{{\partial }^{2}}{\partial {x}^{2}} + \frac{{\partial }^{2}}{\partial {y}^{2}}$$

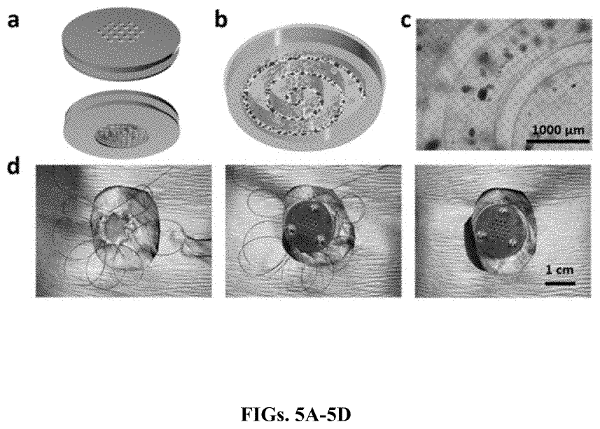

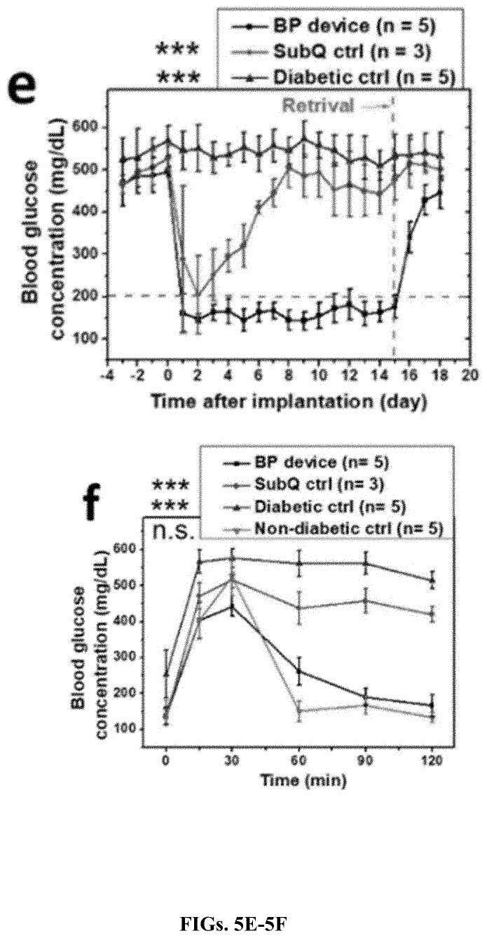

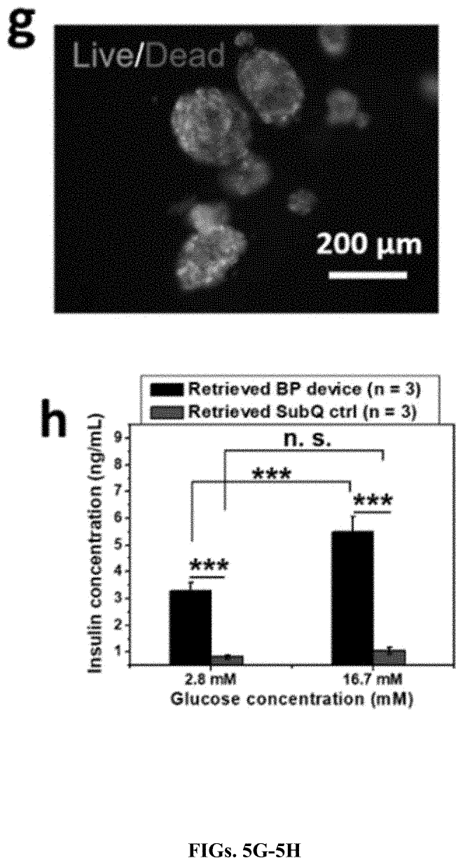

in cartesian coordinates), c_{O}_{₂} represents the concentration of oxygen, and R_{O}_{₂} represents oxygen consumption by encapsulated cells. Resolving Equation 1 for c_{O}_{₂} provides a spatial profile of oxygen concentration within the device. At the air-device interface, it was assumed that the oxygen tension was at atmospheric levels at a partial pressure (pO₂) of 160 mmHg. The concentration at this interface was calculated by multiplying the atmospheric pO₂ by the solubility of oxygen in PFC, α_{O}_{₂}_{,PFC}=0.0254 mM mmHg⁻¹ (Tham, et al., J. Chem. Eng. Data 18:411 (1973); Lewis, Doctor of Philosophy Thesis, Massachusetts Institute of Technology, (2008), which are hereby incorporated by reference in their entirety). At the tissue-device interface, it was assumed that the pO₂ was 24 mmHg, which is a low estimate of subcutaneous oxygen levels according to the literature (Bochenek et al., Nat. Biomed. Eng. 2:810 (2018); Wang et al., J. Physiol. 549:855 (2003), which are hereby incorporated by reference in their entirety). Note, 24 mmHg is equivalent to 3% oxygen, which was the condition set in the hypoxia incubator in the complementary in vitro study (FIG. 5E). The concentration of oxygen was again calculated by multiplying this value with the solubility of oxygen in PFC or alginate, α_{O}_{₂}_{,PFC} and α_{O}_{_{Z}}_{,alginate}=1.24×10⁻³ mM mmHg⁻¹ respectively (Lewis, Doctor of Philosophy Thesis, Massachusetts Institute of Technology, 2008, which is hereby incorporated by reference in its entirety). No-flux boundary conditions were applied at the sides under the assumption that the titanium frame base significantly limited oxygen transport from this boundary. Further, an oxygen partition coefficient,

$${K}_{{O}_{2} , PFCjalginate} = \frac{{α}_{{O}_{2} , PFC}}{{α}_{{O}_{2} , alginate}} = 20.5$$

(equivalent to the solubility ratio), was applied at all PFC-alginate interfaces to reflect gas partitioning. Boundary conditions and dimensions are illustrated in FIG. 9. For both models, literature estimates were used for oxygen diffusivity in the alginate (D_{O}_{₂}_{,alginate}=2.5×10⁻⁹ m²s⁻¹ (Mehmetoglu, et al., Artif. Cells Blood Substit. Immobil. Biotechnol. 24:91 (1996); White et al., Polym. Adv. Technol. 25:1242 (2014); Zhao et al., J. Chem. Technol. Biotechnol. 88:449 (2012); Li et al., Biotechnol. Bioeng. 50:365 (1995), which are hereby incorporated by reference in their entirety) and PFC (D_{O}_{₂}_{,PFC}=5.5×10⁻⁹ m²s⁻¹) (Tham et al., J. Chem. Eng. Data 18:411 (1973), which is hereby incorporated by reference in its entirety) domains.

Model 1 was investigated as a 2D system where islets, implemented as circles, were assumed to have a uniform diameter of 150 μm (Kilimnik et al., Islets 4:167 (2012), which is hereby incorporated by reference in its entirety). Oxygen diffusivity in islet tissue was given from the literature as 2.0×1.0⁻⁹ m²s⁻¹ (Avgoustiniatos et al., Ind. Eng. Chem. Res. 46:6157 (2007), which is hereby incorporated by reference in its entirety). Oxygen consumption, R_{O}_{₂}, was zero in all domains except the islet domain, where it was modeled using Michaelis-Menten kinetics in accordance with similar models and investigation of mitochondrial respiration (Equation 2) (Buchwald, Theor. Biol. Med. Model. 8:20 (2011); Buchwald, Theor. Biol. Med. Model. 6:5 (2009); Buchwald et al., Biotechnol. Bioeng. 115:232 (2018); Papas et al., Adv. Drug Del. Rev. DOI: 10.1016/j.addr.2019.05.002 (2019); Suszynski et al., J. Diabetes Res. 2016:7625947 (2016); Avgoustiniatos, et al., Ann. N. Y. Acad. Sci. 831:145 (1997); Wilson et al., J. Biol. Chem. 263:2712 (1987), which are hereby incorporated by reference in their entirety)

$${R}_{{O}_{2}} = \{ \begin{matrix}0 & , {c}_{{O}_{2}} < {c}_{critical} \\ {V}_{m , {O}_{2} , islet} ( \frac{{c}_{{O}_{2}}}{{c}_{{O}_{2}} + {K}_{m}} ) & , {c}_{{O}_{2}} \geq {c}_{critical}\end{matrix} \qquad ( 2 )$$

Above, V_{m,O}_{₂}_{,islet}=0.034 mM s⁻¹ represents the maximum oxygen consumption rate of islet tissue,^{[5]} Kₘ=1×10⁻³ mM represents the half-maximal concentration,^{[6g]} and c_{critical}=1×10⁻⁴ represents the concentration of oxygen below which islets necrose and cease oxygen consumption (Buchwald, Theor. Biol. Med. Model. 8:20 (2011); Buchwald, Theor. Biol. Med. Model. 6:5 (2009); Buchwald et al., Biotechnol. Bioeng. 115:232 (2018); Papas et al., Adv. Drug Del. Rev. DOI: 10.1016/j.addr.2019.05.002 (2019); Suszynski et al., J. Diabetes Res. 2016:7625947 (2016); Avgoustiniatos, et al., Ann. N. Y. Acad. Sci. 831:145 (1997); Wilson et al., J. Biol. Chem. 263:2712 (1987), which are hereby incorporated by reference in their entirety).

Model 2 was implemented as a 2D axisymmetric system where a composite domain of cells and alginate was considered instead of discretizing each component. The diffusivity of oxygen in alginate, D_{O}_{₂}_{,alginate} was applied in the composite domain due to the low volume fraction of cells considered in this region. It was also assumed that the cells were homogeneously distributed and thus oxygen consumption was uniform throughout this domain, again according to Michaelis-Menten kinetics (Equation 3).

$${R}_{{O}_{2}} = \{ \begin{matrix}0 & , {c}_{{O}_{2}} < {c}_{critical} \\ {V}_{m , {O}_{2} , INS - 1} \cdot ρ ( \frac{{c}_{{O}_{2}}}{{c}_{{O}_{2}} + {K}_{m}} ) & , {c}_{{O}_{2}} \geq {c}_{critical}\end{matrix} \qquad ( 3 )$$

Here, V_{m,O}_{₂}_{,INS1}=5×10⁻⁷ mol s⁻¹ cell⁻¹ found from the literature (Cline et al. Biochem. Biophys. Res. Commun. 415:30 (2011), which is hereby incorporated by reference in its entirety), represents the oxygen consumption rate of INS-1 cells on a cellular basis and ρ=3×10⁶ cells mL⁻¹ represents the encapsulated cell density determined experimentally.

Finite elements for both models were automatically generated by the COMSOL software using the “Free Triangular” setting. A total number of 102,290 and 8,508 degrees of freedom, respectively, for model 1 and model 2 were sufficient to produce results independent of the mesh.

In vitro mass transfer study. INS-1 cells were cultured in RPMI 1640 medium (Gibco) supplemented with 2 mM glutamine (Gibco), 1 mM sodium pyruvate (Gibco), 250 μg mL⁻¹ amphotericin B, 10 mM HEPES (Sigma-Aldrich), 10% (v/v) heat inactivated fetal bovine serum (Gibco), 50 μM β-mercaptoethanol, and 1% penicillin/streptomycin (Gibco). INS-1 cell culture media were added in 12-well plate and de-oxygenated in a hypoxia incubator (3% O₂, 5% CO₂, 37° C.) overnight to achieve a pO₂ of 24 mmHg. INS-1 cells were encapsulated at a density of 3 million cells mL⁻¹ in metal mouse BP devices and alginate slabs (diameter of 8 mm, thickness of 1.3 mm) and placed in the de-oxygenated media. A barrier layer (mineral oil, MitoXpress) was applied at the media-air interface to impede oxygen transport into the media from the atmosphere. The top face of the BP device was exposed to atmosphere while the remainder of the device was submerged in media, whereas the alginate slab was positioned beneath the oil barrier and completely submerged in the media. Then, the plate was transferred to a standard incubator (no O₂ controller, 5% CO₂, 37° C.). After 36 hours of culture, the encapsulated cells were retrieved and characterized by live/dead staining.

Rat Islet Isolation and Purification.

Sprague-Dawley rats weighing around 300 g were used for harvesting pancreatic islets. All rats were anesthetized using 3% isoflurane in oxygen and maintained at the same rate throughout the procedure. Isolation surgeries were performed as previously reported (Lacy et al., Diabetes 16:35 (1967), which is hereby incorporated by reference in its entirety). Briefly, the bile duct was cannulated and the pancreas was distended by an in vivo injection of 10 mL 0.15% Liberase TL (Roche) in M199 media. The pancreas was digested at 37° C. water bath for 28 minutes. The digestion was stopped by adding cold M199 media with 10% heat-inactivated fetal bovine serum and shaking. Digested pancreases were washed twice in the same aforementioned M199 media, filtered through a 450 μm sieve, and then suspended in a Histopaque 1077 (Sigma)/M199 media gradient and centrifuged at 1,700 RCF at 4° C. This gradient centrifugation step was repeated for higher purity. Finally, the islets were collected from the gradient and further isolated by a series of gravity sedimentations, in which each supernatant was discarded after 4 minutes of settling. Purified islets were hand-counted by aliquot under a light microscope. Islets were then washed once with RPMI 1640 media with 10% HIFBS and 1% penicillin/streptomycin and cultured in this medium overnight before further use.

Implantation and retrieval in mice. Immune-competent male C57BL/6 mice were used for implantation and transplantation. To create insulin-dependent diabetic mice, healthy mice were treated with freshly prepared streptozocin (STZ, Sigma-Aldrich) solution (22.5 mg/mL in sodium citrate buffer solution) at a dosage of 150 mg STZ/kg mouse. The blood glucose levels of all mice were retested prior to transplantation. Only mice with non-fasted blood glucose levels above 350 mg/dL were considered diabetic. The diabetic mice were anesthetized with 3% isoflurane in oxygen and their dorsal skin were shaved and sterilized using betadine and 70% ethanol. A 1.5 cm diameter circular section of skin was excised on the dorsum. The skin around the incision was fitted into the side groove of the biphasic devices, and a purse string suture pattern was performed using a non-absorbable nylon suture. After surgery, Elizabethan collars (Kent Scientific) were attached around the mice neck.

For retrieval, the devices were excised along with the surrounding skin, and the incision was closed using 5-0 absorbable polydioxanone (PDS II) sutures.

Static glucose-stimulated insulin secretion (GSIS) assay. Krebs Ringer Bicarbonate (KRB) buffer (2.6 mM CaCl₂.2H₂O, 1.2 mM MgSO₄.7H₂O, 1.2 mM KH₂PO₄, 4.9 mM KCl, 98.5 mM NaCl, and 25.9 mM NaHCO₃ (all from Sigma-Aldrich) supplemented with 20 mM HEPES (Sigma-Aldrich) and 0.1% BSA (Rockland), was prepared and filtered using a vacuum filter unit with 0.22 μm PES Membrane. The islet encapsulation module was incubated in KRB buffer for 2 hours at 37° C., 5% CO₂, and then incubated in KRB buffer supplemented with 2.8 mM or 16.7 mM glucose for 75 minutes. The buffer was collected from each incubation step and after dilution, the insulin concentrations in the collected solutions were measured using an ultrasensitive rat insulin ELISA kit (ALPCO) according to the supplier's protocol.

Implantation and retrieval in dogs. The dogs were premedicated with gylcopyrolate and butorphanol, induced with propofol, and anesthetized with isoflurane and oxygen. The dorsolateral skin of the dog was shaved and prepared for sterile surgery. A 3 cm diameter circular section of skin was excised using a scalpel, the exposed adipose tissue was excised to create a subcutaneous pocket. The device base was placed into the deep subcutaneous space and fastened to superficial muscle fascia by the anchor rings around the frame base using 3-0 polydioxanone suture. Then, a purse-string suture pattern was placed in the dermis to close the skin incision using 3-0 prolene sutures. Implant sites were post-operatively bandaged with clean gauze and plastic bandage material. Following surgeries, the dogs were housed in AAALAC-approved enclosures and allowed daily outdoor access in a yard adjacent to the research facility.

For retrieval, the cell encapsulation module in the frame cap was unscrewed from the frame base and fixed in formalin. The frame base was excised along with the surrounding soft tissues, fixed in formalin, and then embedded in JB-4 2-hydroxyethyl methacrylate plastic (Polysciences, Inc.) for sectioning. After retrieval, the incision was closed using 3-0 absorbable polydioxanone (PDS II) sutures.

Statistical analysis. Results are expressed as raw data or mean±SD. For random BG measurements, a two-way analysis of covariance (ANCOVA) was performed for measurements between day 7 and day 15 (the region in which assumptions for the ANCOVA were satisfied), where treatment group (i.e. diabetic control versus non-diabetic control versus subcutaneous control) were considered discrete factors and time was considered a continuous covariate. Including all data between day 1 and day 15 did not change the significance conclusion of the test. For the IPGTT test (FIGS. 5E-5F), data was evaluated by a two-way analysis of variance (ANOVA), where treatment (i.e. diabetic control, non-diabetic control, subcutaneous control, and BP device) and time were considered discrete factors, followed by a Tukey post-hoc test. A two-way ANOVA was also used to evaluate data from the GSIS test (FIG. 51I), where treatment (subcutaneous control versus BP device) and buffer concentration (2.8 mM versus 16.7 mM) were considered discrete factors, followed by a Sidak's multiple comparison test. R software was used to perform statistical analyses. Statistical significance was concluded at P<0.05.

### Example 1—Design and Structure of the Cell Replacement Device

The atmosphere is a virtually unlimited source of highly concentrated oxygen. At sea level, the partial pressure of oxygen (pO2) in the atmosphere is ˜160 mmHg—roughly 4 times higher than in common transplantation sites. In fact, the human cornea, which is avascular, is oxygenated by direct contact with the air (Fatt et al., in Physiology of the Eye (Second Edition), (Eds: I. Fatt, B. A. Weissman), Butterworth-Heinemann, 1992, 151, which is hereby incorporated by reference in its entirety). Notably, the aqueous and cellular components of the cornea are protected from evaporation and environmental harm by a lipid/oil-containing layer of the tear film at the atmospheric interface (Cwiklik, Biochim. Biophys. Acta, Biomembranes 1858:2421 (2016); Bron et al., Exp. Eye Res. 78:347 (2004); Mishima et al., Exp. Eye Res. 1:39 (1961); Goto et al., Invest. Ophthalmol. Vis. Sci. 2:533 (2003), which are hereby incorporated by reference in their entirety).

Inspired by this clever natural oxygen delivery strategy, a novel modular biphasic (BP) system was designed. Cells were encapsulated in a hydrogel (liquid phase) and oxygen supply was provided by contact with the atmosphere (gas phase). In mimicry of the cornea, the device was implanted in a transcutaneous position, thereby exposing one face to the air and the other to the subcutaneous space. Further, environmental protection was provided by a perfluorinated carbon (PFC) oil-infused film at the atmospheric interface, such as the role of the surface layer of the tear film. (FIG. 1). The BP device consisted of four fundamental components (FIG. 1A): (1) the PFC cover to provide environmental protection and prevent dehydration, (2) PFC channels within the cell encapsulation domain for improved oxygen transport and mechanical reinforcement, (3) a hydrogel for cell encapsulation and immunoisolation, and (4) a frame to fasten the device in a transcutaneous configuration. Importantly, the cell encapsulation module was attached to a demountable cap, which allowed for the replacement of the therapeutic cells within a few minutes non-surgically. Eventual graft decline may necessitate additional transplantations for sustained therapeutic activity. The modularity of the BP device provided a platform to circumvent complicated and invasive surgical procedures traditionally required for graft replacement.

The characterization and testing of the BP device is presented herein. Simulation and in vitro investigations show improved graft oxygenation in comparison to subcutaneous transplantation. Encapsulated pancreatic islet transplantation—a promising application of cell replacement therapy—offers to improve type 1 diabetes treatment by eliminating or reducing the need for exogenous insulin injections (Weir, Diabetologia 56:1458 (2013); Scharp et al., Adv. Drug Del. Rev. 67-68:35 (2014); Desai et al., Nat. Rev. Drug Discov. 16:338 (2017); Ernst et al., J. Mater. Chem. B 6:6705 (2018), which are hereby incorporated by reference in their entirety). Islets (cell clusters between tens and hundreds of microns in diameter containing hundreds to thousands of insulin-secreting β cells and other secretory cell types) are particularly vulnerable to hypoxia (Dionne et al., Diabetes 42:12 (1993), which is hereby incorporated by reference in its entirety). Stripped of their native microvasculature during isolation (Bowers et al., Acta Biomater. DOI: 10.1016/j.actbio.2019.05.051 (2019), which is hereby incorporated by reference in its entirety) islets maintain a steep oxygen consumption rate due to the high metabolic demand of insulin secretion (Avgoustiniatos et al., Ind. Eng. Chem. Res. 46:6157 (2007), which is hereby incorporated by reference in its entirety) and their low capacity for anaerobic respiration (Papas et al., Ann. N. Y. Acad. Sci. 944:96 (2001), which is hereby incorporated by reference in its entirety). Moreover, the capacity of islets to secrete insulin in response to glucose is significantly reduced at even moderately low oxygen levels (Dionne et al., Diabetes 42:12 (1993), which is hereby incorporated by reference in its entirety) and after acute exposure to hypoxia (Smith et al., Transplantation 101:2705 (2017), which is hereby incorporated by reference in its entirety). Therefore, rat islet-encapsulating BP devices were tested in immunocompetent streptozotocin (STZ)-induced diabetic mice and confirmed that the device was able to maintain islet health and provide diabetes correction in vivo. Finally, robust cell survival and a proof of concept of the cell refilling procedure was shown in dogs.

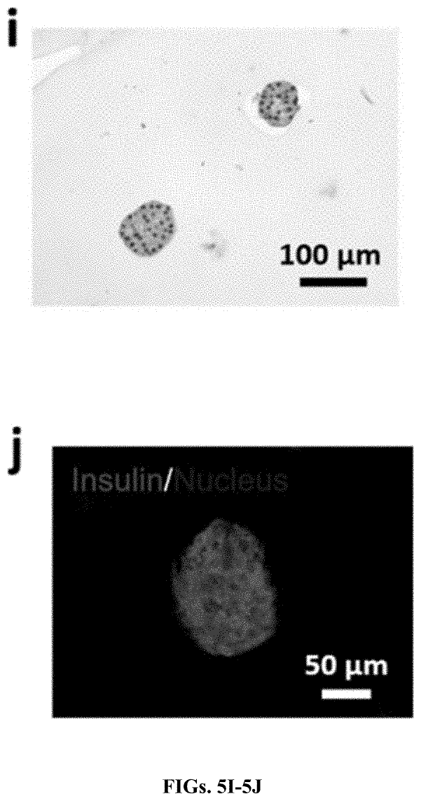

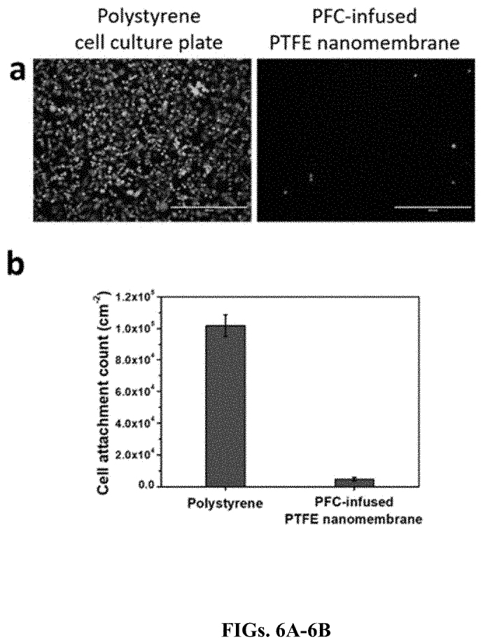

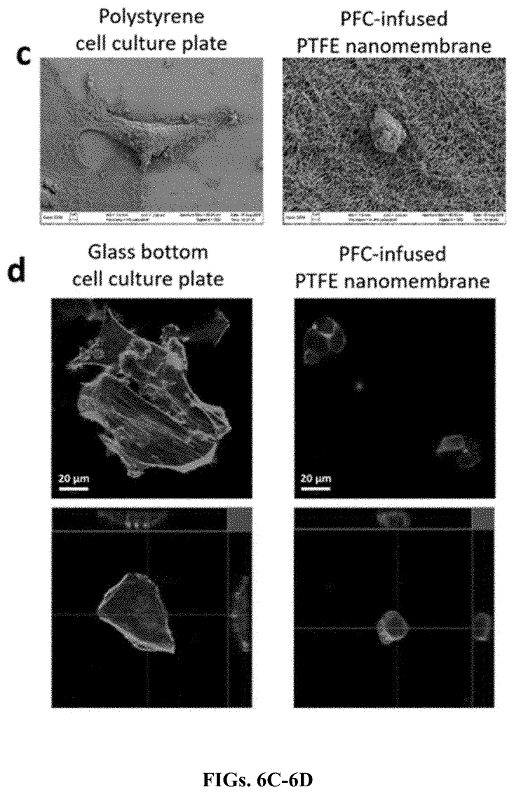

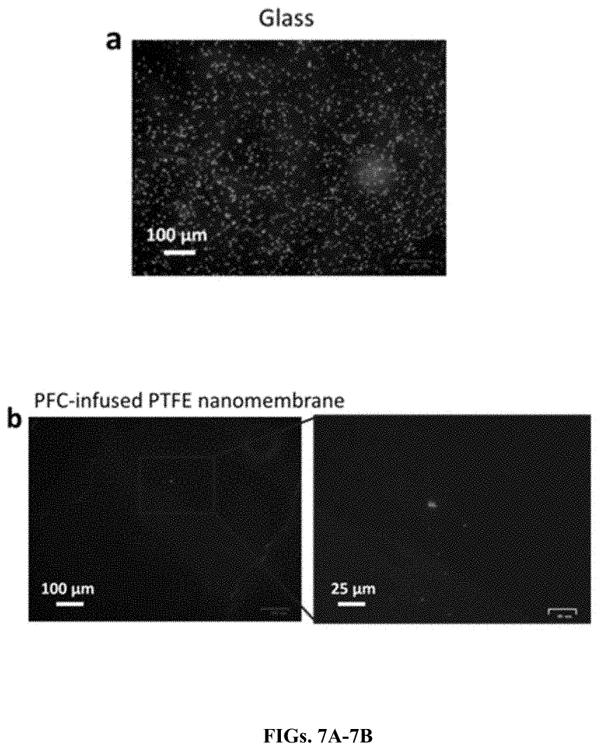

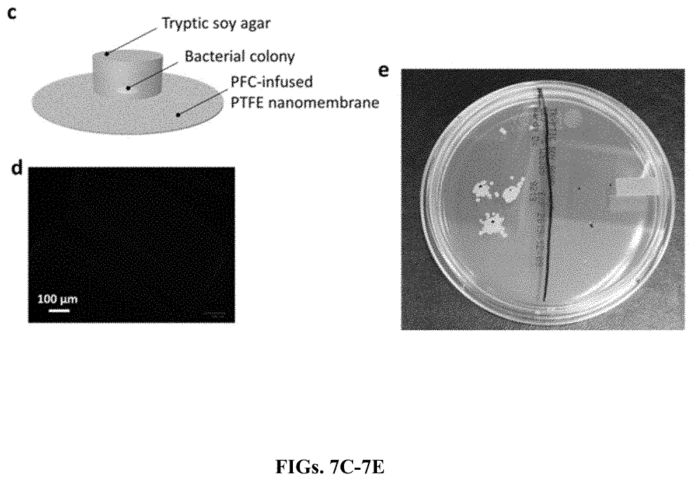

First, a prototype was designed for implantation in a mouse (FIGS. 1B-1C). The frame of this device, comprising both a base and a cap, was composed of titanium for its well-documented biocompatibility (Sidambe, Materials (Basel) 7:8168 (2014), which is hereby incorporated by reference in its entirety). Several portals (1 mm diameter) were fabricated into the cap to allow gas exchange with the atmosphere, whereas the bottom face of the base was open as to totally expose the graft to the subcutaneous tissue to ensure nutrient exchange between the host and the encapsulated cells. A PFC (Krytox®, GPL103)-infused polytetrafluoroethylene (PTFE) nanomembrane was applied below the titanium cap at the device-atmosphere interface (FIG. 1D). The low surface energy and nanoporosity (˜200 nm pore size) of the PTFE mesh created a strong capillary force that enabled PFC infiltration and retention. Prior investigation demonstrated that bacterial adhesion to this membrane was severely limited (Chen et al., Biomaterials 113:80 (2017), which is hereby incorporated by reference in its entirety). Moreover, the mesh pore size corresponded to that of standard bacterial filtration membranes, therefore the membrane was thus expected to prevent both the adhesion and passage of bacteria through this interface. In addition, this composite material was non-wettable, non-volatile, and omniphobic (Wong et al., Nature 477:443 (2011); Leslie et al., Nat. Biotechnol. 32:1134 (2014), which are hereby incorporated by reference in their entirety) and was accordingly an optimal material for both barring environmental stressors from graft interference and preventing hydrogel dehydration (FIGS. 1E-1F). Cell and bacterial culture on the PFC-infused PTFE membrane also showed significantly impaired adhesion, though cell viability was preserved, suggesting that this material would both reject bacterial infiltration while providing no harm to the encapsulated cells (FIG. 6 and FIGS. 7A-7B). Furthermore, an in vitro test suggested that bacterial migration through the membrane was prohibited (FIGS. 7C-7E).

Additionally, a spiral poly(vinylidene fluoride-co-hexafluoropropylene) (PVDF-HFP) scaffold (FIG. 1G-1H) was fabricated directly on the PTFE membrane. This fluorinated polymer scaffolding was porous, which allowed for PFC infiltration and thus provided both structural reinforcement and improved oxygen delivery. Around the scaffolding, a suspension of cells within ultrapure sodium alginate (Pronova SLG100) was added via pipet and crosslinked by submersion in a 95 mM CaCl₂) and 5 mM BaCl₂ buffer. Alginate was selected as the cell encapsulation hydrogel for its biocompatibility and common application in cell encapsulation (Lee et al., Prog. Polym. Sci. 37:106 (2012); Orive et al., in Immobilization of Enzymes and Cells, (Ed: J. M. Guisan), Humana Press, 345 (2006), which are hereby incorporated by reference in their entirety). A cell-free alginate-impregnated nylon mesh (70 μm thickness) was applied for mechanical reinforcement at the device-host interface (FIG. 1I-1J). The above components constituted the cell encapsulation module (FIG. 4). Finally, a PDMS O-ring was included between the cell encapsulation module and the frame base to ensure a tight seal.

### Example 2—Improved Graft Oxygenation to Cells of the Cell Replacement Device

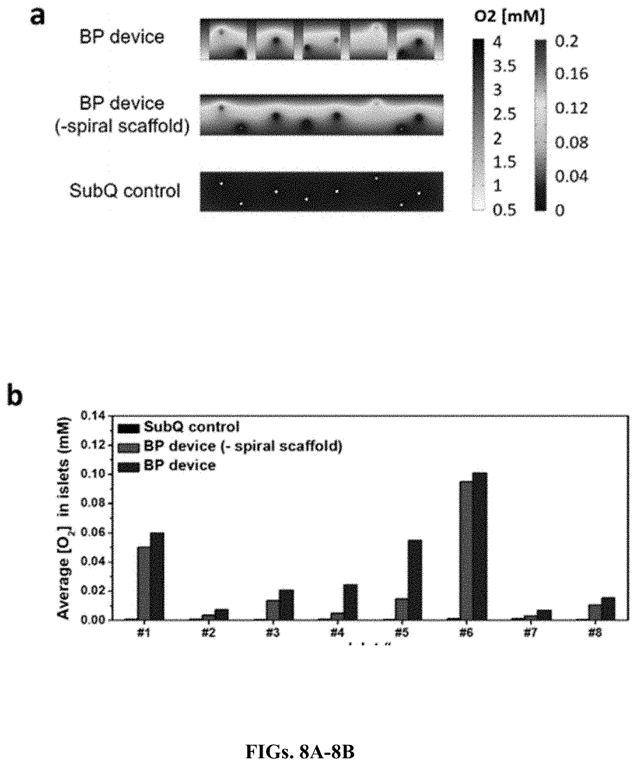

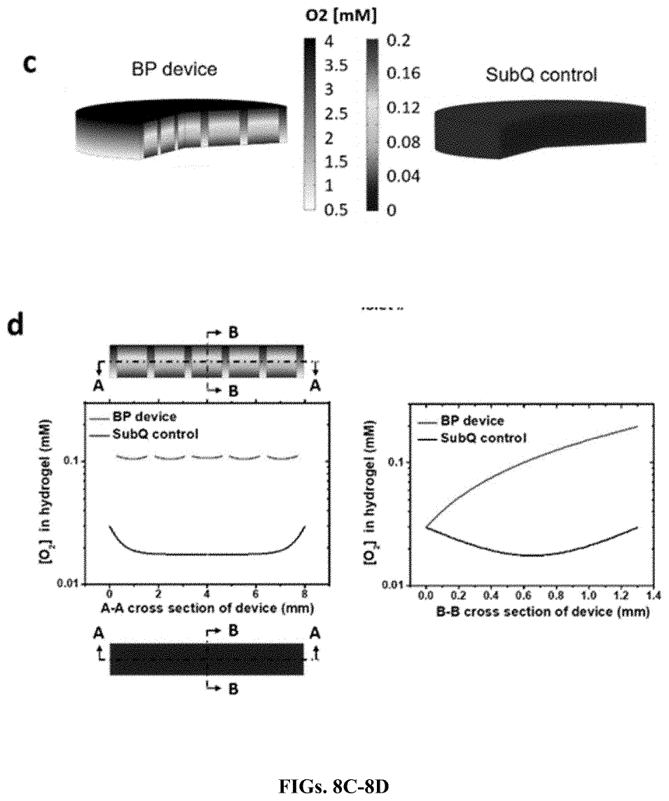

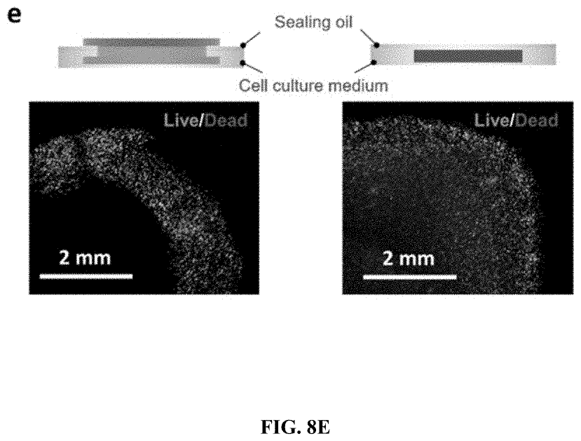

Subsequently, the oxygen transfer advantages of the transcutaneous concept were investigated by theoretical and in vitro analyses (FIG. 8). Computational models were developed to compare the oxygenation of randomly seeded islets in the “transcutaneous” BP device, the “transcutaneous” device without the spiral scaffold, and a “subcutaneous” control (see the Mass Transfer section supra and FIG. 9). The difference between the subcutaneous and transcutaneous configurations was implemented by applying a top boundary condition of oxygen tension of 24 mmHg (3% oxygen) or 160 mmHg (21% oxygen), corresponding with subcutaneous and atmospheric levels respectively (the boundary condition at the device-subcutaneous space interface was 24 mmHg for both conditions).

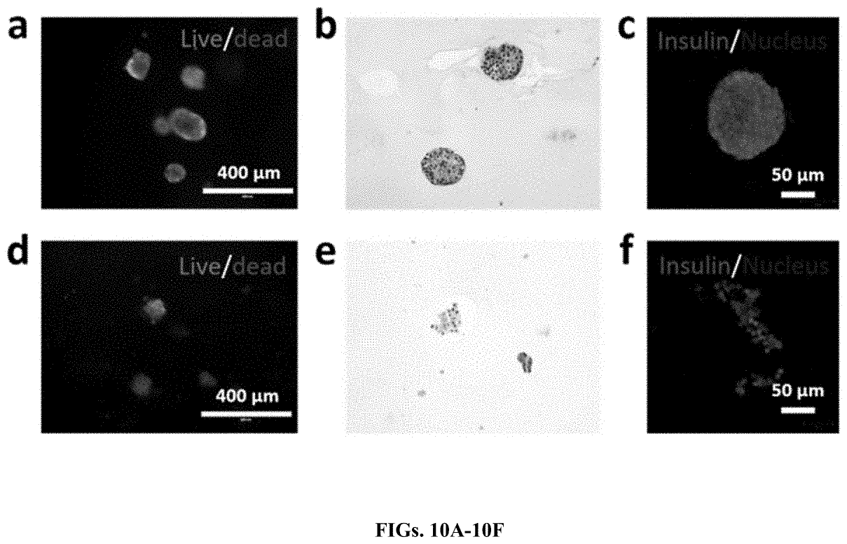

Simulation revealed that the BP device provided significantly higher predicted oxygenation in the alginate-cell domain in comparison to the subcutaneous control. The concentration of oxygen in the PFC film was also predicted to be higher than that within the hydrogel due to its superior oxygen solubility. Most importantly, robust islet survival was predicted for the BP device, whereas a high degree of islet necrosis was predicted in the subcutaneously implanted control (FIG. 8A and FIG. 10). Further, quantification of spatially averaged oxygen concentration within individual islets suggested that the inclusion of the spiral scaffold modestly to significantly improved islet oxygenation, depending on the proximity of the cell cluster to the scaffold (FIG. 8B). This result, in addition to the qualitatively noted improved mechanical strength, encouraged the incorporation of the spiral scaffold in all ensuing testing.

Another model was developed to simulate oxygen transport in a dispersed cell encapsulation system. Again, significantly higher graft oxygenation was predicted for the BP device in comparison to the subcutaneous control (FIG. 8C). Along a horizontal cross section, predicted graft oxygenation was nearly an order of magnitude higher in the BP device compared to the control; along a vertical cross section, the highest oxygen concentration of the control device represented the lowest oxygen concentration of the BP device (FIG. 8D). These simulated predictions were validated by an in vitro analysis. INS-1 cells were encapsulated at a density of 3 million cells mL⁻¹ in a BP device and an alginate slab and placed in media which had been previously reduced to a pO₂ of 24 mmHg (3% oxygen) in a hypoxia chamber. A barrier layer (MitoXpress oil) was applied at the media-air interface to impede oxygen transport into the media from the atmosphere. The top face of the BP device was exposed to the atmosphere while the remainder of the device was submerged in media, whereas the alginate slab was positioned beneath the oil barrier and completely submerged in the media. Robust cell survival was observed in the transcutaneously-positioned BP device, whereas only an outer layer of viable cells remained in the subcutaneous control (FIG. 8E). These studies and analyses corroborated the modeling results and reaffirmed our hypothesis that exposing the device to the atmosphere would significantly improve graft oxygenation.

### Example 3—Therapeutic Utility of Cell Replacement Device

The therapeutic capability of the BP device was next tested in a rat-to-mouse xenotransplantation model (FIG. 5). Mouse devices encapsulating isolated rat islets (500 islet equivalents; IEQ) within ˜50 μL of alginate, were fabricated for transcutaneous transplantation in STZ-induced diabetic C57BL/6J mice (FIGS. 5A-5D). Hyperglycemia reversal (blood glucose, BG<200 mg dL⁻¹) was observed after 1 day and for the duration of the study (15 days) in animals treated with the BP device. Brief BG lowering was observed in subcutaneously transplanted alginate slab controls encapsulating 500 IEQ rat islets (see FIG. 9B), though the mice returned to a hyperglycemic state (BG>450 mg dL⁻¹) within 1 week following transplantation. Diabetic controls were hyperglycemic at all readings over the course of the study.

An intraperitoneal glucose tolerance test (IPGTT) was performed on the mice at day 7 to further test device function. The BG of the BP device-treated group returned to a lowered state after 60 minutes, which was similar to healthy controls, whereas the blood glucose of the subcutaneously implanted controls did not lower over the 120 minutes investigated, similar to the diabetic controls (FIG. 5F). Live/dead staining of retrieved islets showed that the encapsulated cells in BP devices were largely viable following retrieval (FIG. 5G). Furthermore, a static GSIS performed on the retrieved cell encapsulation modules and subcutaneous controls showed glucose responsiveness of the BP devices, whereas insulin secretion was significantly impaired in the retrieved subcutaneous controls (FIG. 5H). Maintained islet function in the BP device was further corroborated by healthy islets found in hematoxylin and eosin (H&E) stained slides and the robust presence of insulin following immunostaining (FIG. 5I-5J). In contrast, islet health was significantly impaired in retrieved subcutaneous samples (FIG. 10).

### Example 4—Scale-Up and Refilling of Cell Replacement Devices

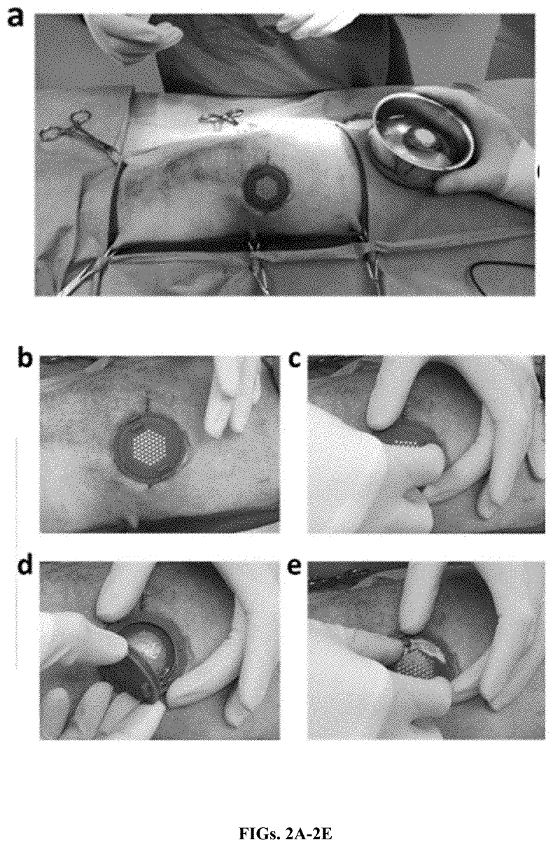

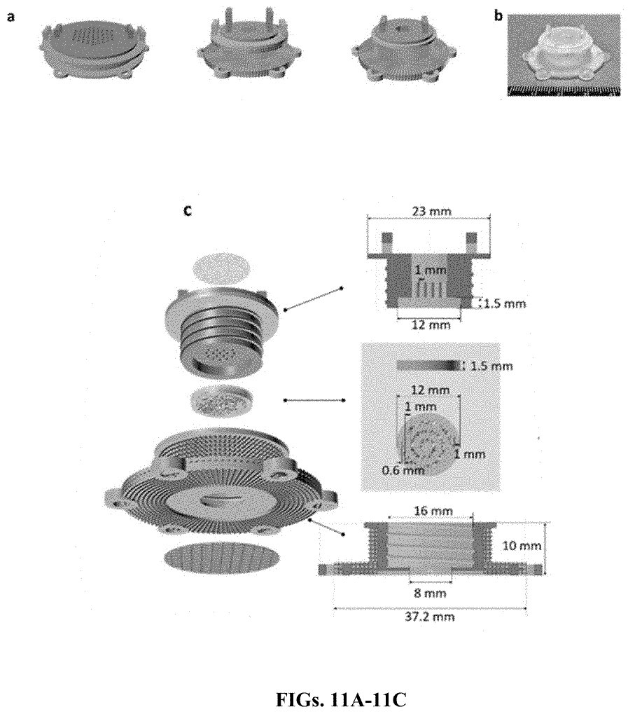

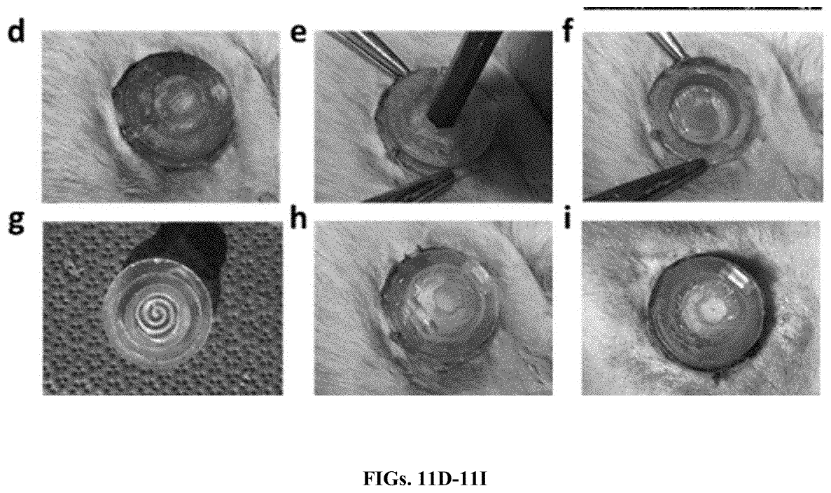

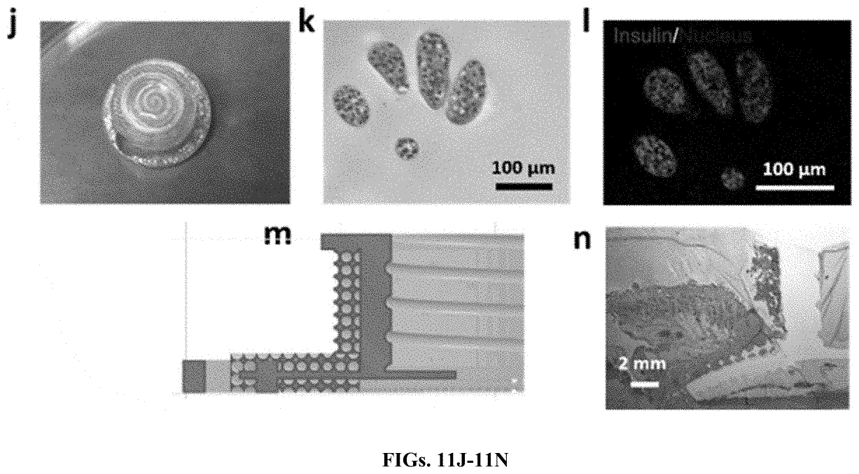

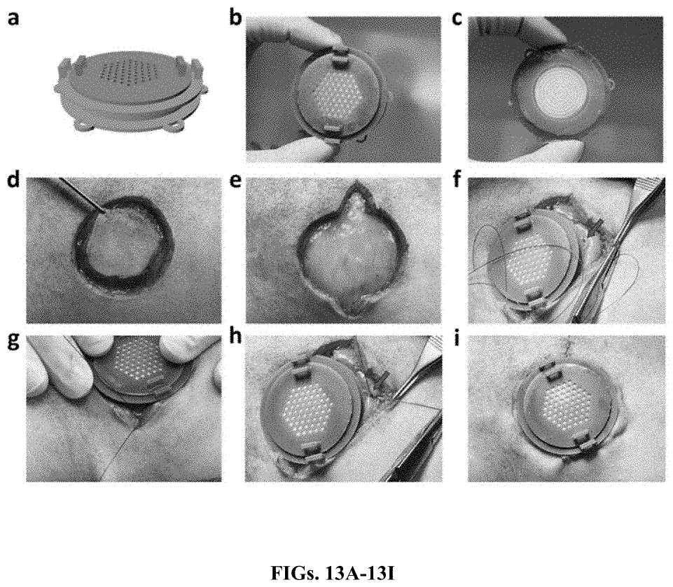

The engineering of a BP device for large animal transplantation and cell refilling was subsequently investigated (FIG. 11). A series of design iterations were pursued to overcome translational hurdles (the evolution of the design is illustrated in FIG. 11A and FIG. 12). While the fundamental components of the BP device were preserved, the final design featured several new functionalities inspired by iterative analysis (FIG. 11B-11C). Instead of titanium, the frame was fabricated by 3-dimensional (3D) printing (Form2 3D printer) with the Class IIa biocompatible Dental LT resin as this material provided greater flexibility over design modifications. On the frame base, six anchor rings were incorporated to fasten the device within the subcutaneous tissue via suturing; this was motivated by the successful application of this technique in a dog in the first design (FIG. 13). Testing of the first design in dogs also showed that poor device fixation led to infection (FIG. 12D and FIG. 2).

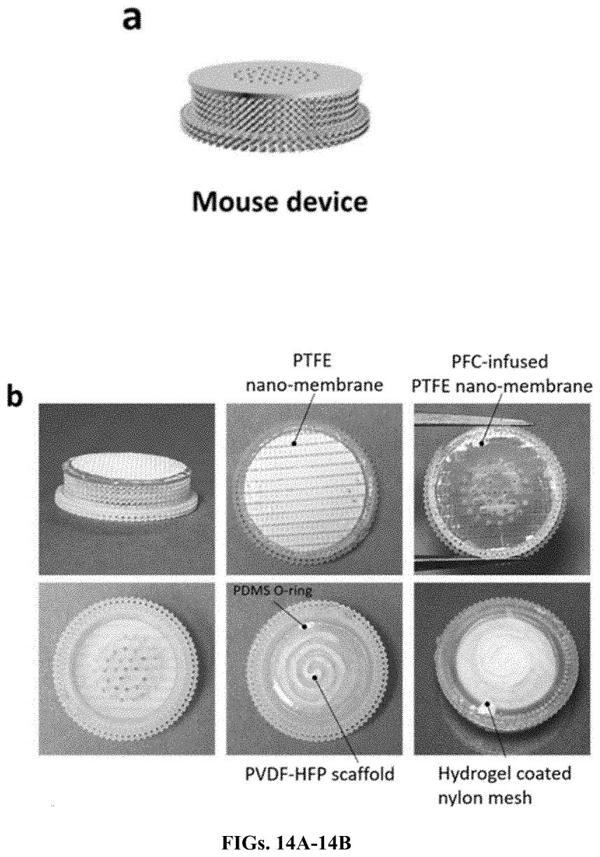

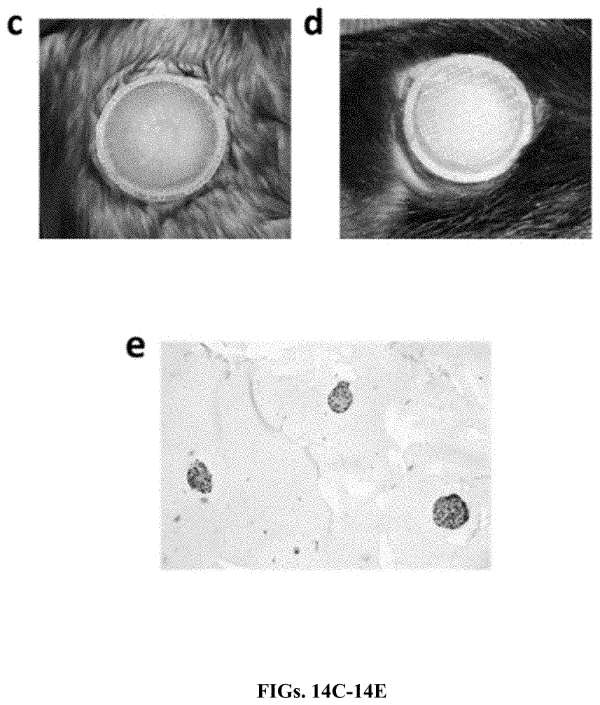

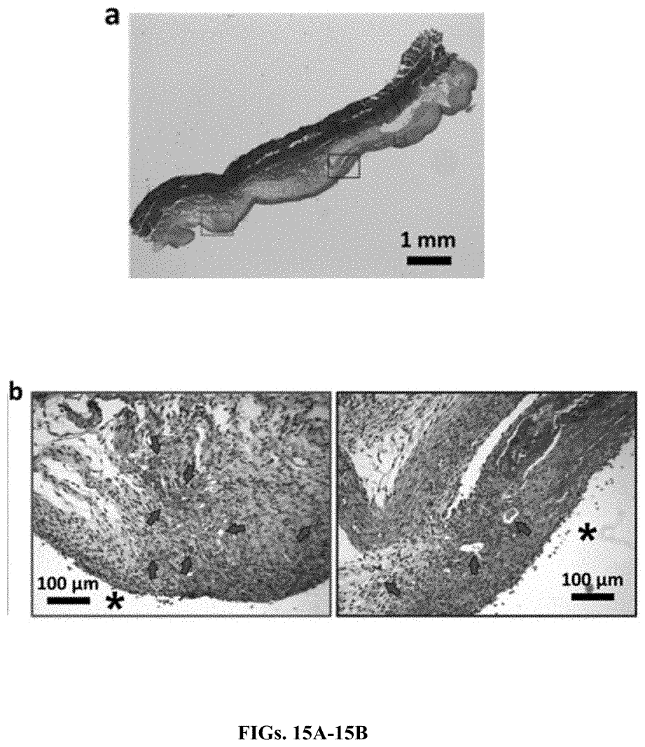

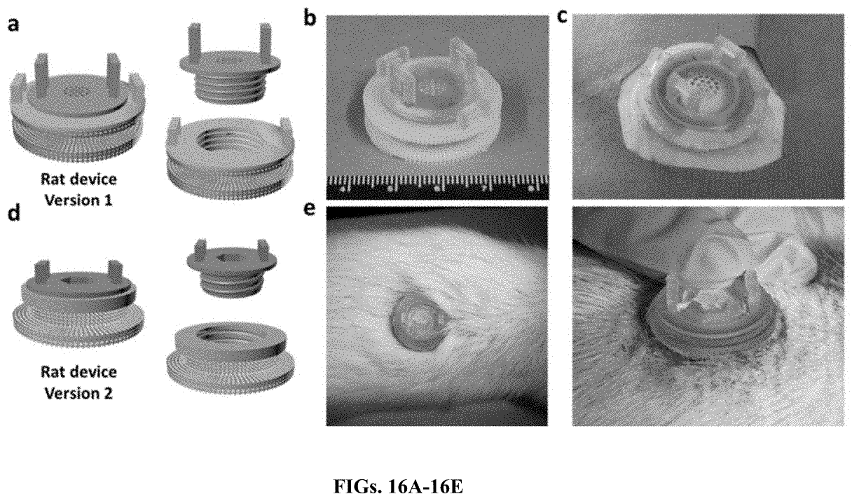

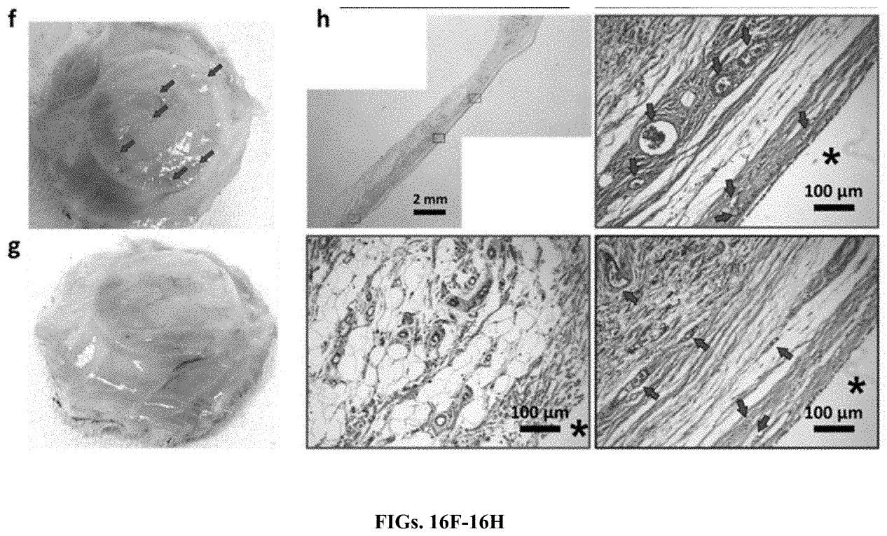

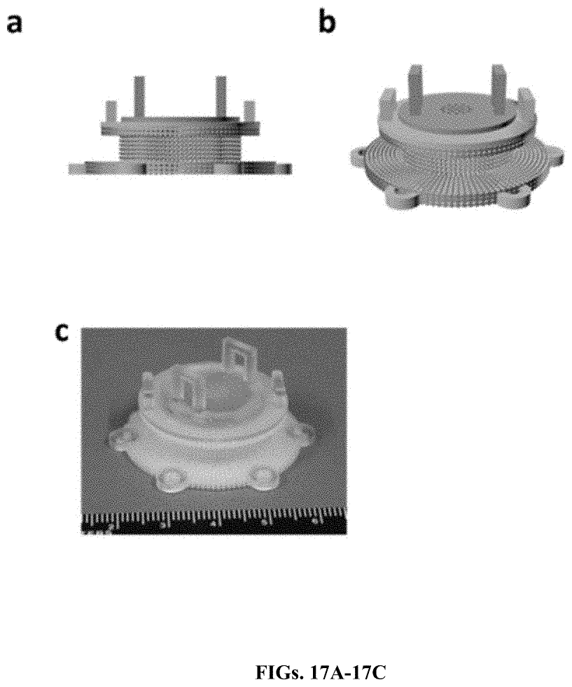

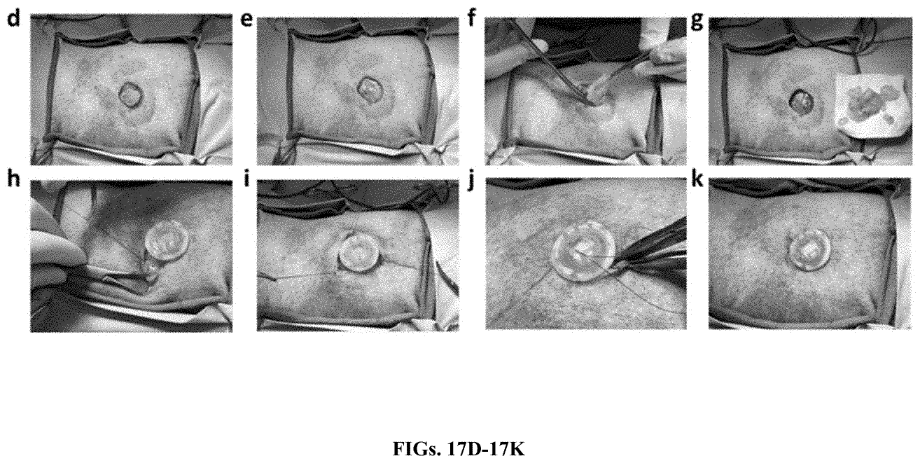

A macroporous structure was therefore incorporated on the frame base, which resulted in robust tissue ingrowth and the transcutaneous fixation of the new device design in mice, rats, and dogs (FIGS. 14-17) for over 1 month. This was consistent with another finding in the literature that demonstrated that porous implants improved tissue integration in the subcutis and cutis, which was further hypothesized to lower the risk of bacterial infection (Hugate et al., Int. J. Adv. Mater. Res. 1:32 (2015), which is hereby incorporated by reference in its entirety). Furthermore, the frame height was increased such that the bottom face of the cell encapsulation module was exposed to the deep subcutaneous tissue following the excision of the cutis and some subcutaneous adipose tissue (FIGS. 17F-17G). Implantation in this region was desirable as it has been suggested that oxygen levels are higher in the deep subcutaneous space in comparison to superficial regions of the tissue (Wang et al., J. Physiol. 549:855 (2003); Carreau et al., J. Cell. Mol. Med. 15:1239 (2011), which are hereby incorporated by reference in their entirety). The foreign body response at the interface of the alginate-impregnated nylon mesh and the host subcutaneous tissue in both mice and rats was characterized by immunohistochemical staining, revealing a slightly vascularized collagenous and cellular (FIG. 15, FIGS. 16F-16H).

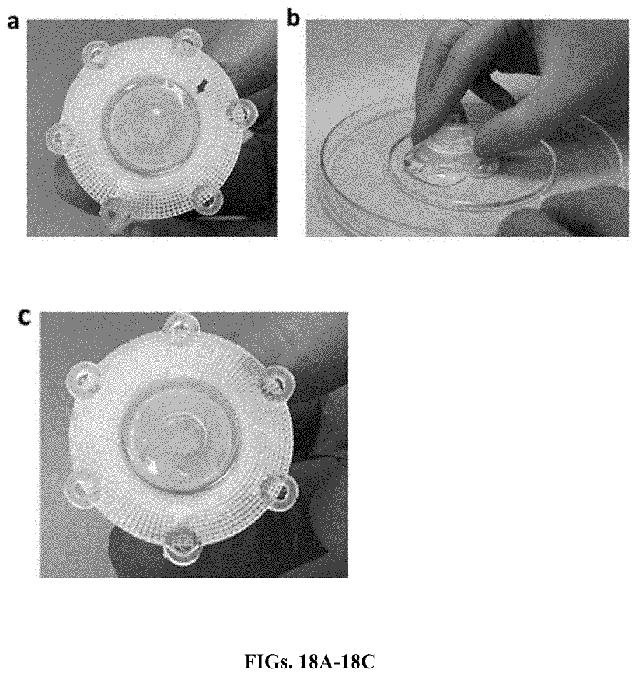

A simple approach was implemented to allow cell refilling. Threading was included on the frame cap and base, and therefore the cap could be removed and replaced by counterclockwise and clockwise rotation respectively. The cell encapsulation module was attached to the frame cap by the inclusion of a PDMS O-ring, as in the previous design; thus, cell refilling was performed by unscrewing the current cap and replacing it with a new one. In the final design, a hexagon depression was integrated into the frame cap such that this process could be performed more easily using a hex wrench (i.e. Allen wrench). In addition, robust attachment of the alginate-impregnated nylon mesh was achieved by situating it within a small depression in the bottom of the frame base and allowing the infiltration of some alginate into adjacent macropores prior to gelation (FIG. 18). Lastly, an additional PFC-impregnated PTFE nanomembrane was placed on top of the frame cap for increased environmental protection, and a PDMS washer was included between the frame base and cap to ensure sealing (FIG. 19).

The BP device was tested in a healthy dog. A cell-free BP device was transcutaneously implanted by the method described above. While the surgical procedures were performed under sterile conditions, animals were kept in AAALAC-approved non-sterile enclosures and allowed daily outdoor access, thus the graft was exposed to potential environmental stressors. At 1 month following transplantation, device integration was robust, and no adverse reaction was observed (FIG. 11D). Using a hex wrench, the frame cap was unscrewed, and the (cell-free) cell encapsulation module was removed (FIGS. 11E-11F). The retrieved cell encapsulation module was not infected or noticeably affected by either the environment or the immune system according to qualitative observation (FIG. 11G). Next, a new cell encapsulation module containing encapsulated rat islets (2000 IEQ in ˜75 μL alginate) was attached to the frame cap and twisted manually (i.e. non-surgically) into the frame base at the transplantation site (FIG. 11H). The removal-and-replacement procedure was performed in a few minutes.

At 1 month following cell refilling, the device maintained tissue integration and structural integrity (FIG. 11I). The cell encapsulation module was removed by the same mechanism described above (FIG. 11J). Retrieved encapsulated islets were found to be mostly healthy and insulin positive as confirmed by H&E and immunohistochemical staining (FIGS. 11K-11L). H&E-stained slides of the excised frame base and surrounding tissue revealed robust tissue ingrowth into the negative space of the macroporous structure (FIGS. 11M-11N). This study demonstrated the feasibility of replacing the transplanted cells when necessary without surgical intervention.

### Discussion of Examples

While several groups have reported improved graft oxygenation, there are a few salient advantages of the biphasic system worth reiterating. Foremost, continuous contact with the atmosphere does not require patient intervention nor the introduction of oxygen generating technologies. Nonetheless, as atmospheric contact exposes the device to environmental harm and bacterial contamination, two key materials strategies were employed to overcome this challenge. First, the application of the omniphobic PFC-infused PTFE nanomembrane was critical for avoiding infection through the portals of the frame cap. It has also been hypothesized that tissue ingrowth facilitates the introduction of immune components into the device-tissue interface, which act to resist bacterial passage (Hugate et al., Int. J. Adv. Mater. Res. 1:32 (2015), which is hereby incorporated by reference in its entirety). Therefore, the incorporation of the porous structure on the exterior of the frame base, which was demonstrated to encourage tissue ingrowth, may have played an equally important role in preventing infection.

Cell replacement therapies have the potential to shift the paradigm of chronic disease treatment, although this technology is often constrained by limited oxygen supply and the difficulty of refilling the therapeutic cells after graft decline. In this report, the design, engineering, and testing of a highly oxygenated biphasic cell encapsulation platform, which enabled gas exchange by contact with the atmosphere and supported cell refilling without requiring surgery, is presented. These benefits were realized by rational design, where immune protection and cell survival were accomplished by hydrogel encapsulation and environmental protection was facilitated by a PFC oil-infused nanomembrane interface. High oxygenation in comparison to subcutaneously implanted grafts was confirmed by theoretical analysis and in vitro studies. Moreover, the therapeutic efficacy of this device was shown in a rat-to-mouse xenograft. Finally, non-surgical cell refilling was shown in a transcutaneous canine implantation. The continued investigation of practical solutions for persisting problems in cell encapsulation will contribute to translation of such devices to the clinic.

Although preferred embodiments have been depicted and described in detail herein, it will be apparent to those skilled in the relevant art that various modifications, additions, substitutions, and the like can be made without departing from the spirit of the present application and these are therefore considered to be within the scope of the present application as defined in the claims which follow.

What is claimed:

1. A cell replacement device, comprising:
a frame cap including a first connecting member and including one or more ports traversing a thickness of the frame cap;
a frame base including one or more walls defining an interior chamber, defining a first opening to the interior chamber on one side of the frame base and defining a second opening to the interior chamber on another side of the frame base, the first opening configured to receive the frame cap, the frame base further including a second connecting member constructed to connect with the first connecting member, wherein at least a portion of at least one of the one or more walls is macroporous; and
a mesh disposed adjacent the second opening.

2. A device comprising a cell encapsulation module, said cell encapsulation module comprising:
a nanomembrane substrate; and
a porous scaffold extending from the nanomembrane substrate.

3. A cell replacement device extending longitudinally from a first end to a second end, said cell replacement device comprising:
(i) a cell encapsulation module, said module comprising a nanomembrane substrate and a porous scaffold extending from a surface of the nanomembrane substrate;
(ii) a frame cap proximal to the first end of the device, said frame cap comprising:
a first surface and a second surface, said second surface proximal to the nanomembrane substrate of the cell encapsulation module, and
one or more ports traversing the thickness of the frame cap through the first and second surfaces of the frame cap; and
(iii) a frame base proximal to the second end of the device, said frame base comprising:
one or more walls defining an interior chamber defining a first opening to the interior chamber on one side of the frame base, and defining a second opening to the interior chamber on another side of the frame base, the first opening configured to receive the frame cap, wherein at least a portion of at least one of the one or more walls is macroporous, and
a mesh disposed adjacent to the second opening of the frame base, wherein the frame base is coupled to the frame cap to form a housing that surrounds the cell encapsulation module.

4. The device of claim 3, wherein the nanomembrane substrate of the cell encapsulation module is infused with perfluorinated carbon oil, silicon oil, or mineral oil.

5. The device of claim 3, wherein the nanomembrane substrate comprises a polytetrafluoroethylene (PTFE) nanomembrane.

6. The device of claim 3, wherein the nanomembrane substrate comprises a non-fluorinated polymer chemically modified with fluoroalkysilanes.

7. The device of claim 3, wherein the nanomembrane substrate has a nanoporosity of between 50 and 500 nm.

8. The device of claim 3, wherein the porous scaffold of the cell encapsulation module comprises a fluorinated polymer material.

9. The device of claim 8, wherein the fluorinated polymer material is poly(vinylidene fluoride-co-hexafluoropropylene) (PVDF-HFP)

10. The device of claim 3, wherein the porous scaffold of the cell encapsulation module comprises a non-fluorinated polymer material chemically modified with fluoroalkysilanes.

11. The device of claim 3, wherein the frame cap and frame base are composed of a non-dissolvable, biocompatible material.

12. The device of claim 11, wherein the non-dissolvable, biocompatible material is titanium or a synthetic resin.

13. The device of claim 11, wherein the non-dissolvable, biocompatible material is a polycaprolactone, poly(lactic acid) material.

14. The device of claim 3, wherein the mesh disposed of adjacent to the second opening in the frame base is a nylon mesh.

15. The device of claim 14, wherein the nylon mesh is coated with a hydrogel material.

16. The device of claim 15, wherein the hydrogel material is natural polymer hydrogel material.

17. The device of claim 16, wherein the natural polymer hydrogel material is selected from the group consisting of alginate, collagen, hyaluronate, fibrin, fibroin, agarose, chitosan, bacterial cellulose, elastin, keratin, and combinations thereof.

18. The device of claim 16, wherein the natural polymer hydrogel material is a zwitterionically-modified natural hydrogel material.

19. The device of claim 15, wherein the hydrogel material is a synthetic polymer hydrogel material.

20. The device of claim 19, wherein the synthetic polymer hydrogel material is polyethylene glycol, a polyethylene glycol derivative, poly(2-hydroxyethyl methacrylate), a poly(2-hydroxyethyl methacrylate) derivative, or combinations thereof.

21. The device of claim 3, wherein the frame base comprises one or more anchor rings suitable for fastening the device to a biological substrate.

22. The device of claim 3, wherein the frame cap is detachable from the frame base.

23. The device of claim 3, wherein said device further comprises a nanomembrane film covering a portion of the first surface of the frame cap.

24. The device of claim 23, wherein the nanomembrane film is infused with perfluorinated carbon oil, silicon oil, or mineral oil.

25. The device of claim 3 further comprising:
a preparation of cells suspended in a hydrogel material within the cell encapsulation module.

26. The device of claim 25, wherein the hydrogel material is a natural polymer hydrogel material.

27. The device of claim 26, wherein the natural polymer hydrogel material is selected from the group consisting of alginate, collagen, hyaluronate, fibrin, fibroin, agarose, chitosan, bacterial cellulose, elastin, keratin, and combinations thereof.

28. The device of claim 26, wherein the natural polymer hydrogel material is a zwitterionically-modified natural polymer hydrogel material.

29. The device of claim 25, wherein the hydrogel material is a synthetic polymer hydrogel material.

30. The device of claim 29, wherein the synthetic polymer hydrogel material is polyethylene glycol, a polyethylene glycol derivative, poly(2-hydroxyethyl methacrylate), a poly(2-hydroxyethyl methacrylate) derivative, or any combinations thereof.

31. The device of claim 25, wherein the preparation of cells is a preparation of any one or more of endothelial cells, smooth muscle cells, cardiac muscle cells, cardiac myocytes, epithelial cells, urothelial cells, fibroblasts, myoblasts, chondrocytes, chondroblasts, osteoblasts, keratinocytes, hepatocytes, renal cells, pulmonary cells, bile duct cells, pancreatic islet cells, thyroid cells, parathyroid cells, adrenal cells, hypothalamic cells, pituitary cells, ovarian cells, testicular cells, salivary gland cells, adipocytes, embryonic stem cells, adult stem cells, induced pluripotent stem cells, mesenchymal stem cells, neuronal cells, astrocytes, oligodendrocytes, hematopoietic cells, and any precursor or progenitor cell thereof.

32. The device of claim 25, wherein the preparation of cells is a preparation of cells engineered to recombinantly express a therapeutic agent.

33. The device of claim 25, wherein the preparation of cells is a preparation of pancreatic islet cells.

34. A method for delivering a therapeutic agent to a subject in need thereof, said method comprising:
providing the cell replacement device of claim 3; and
implanting the cell replacement device transcutaneously into a region of the subject suitable for delivering the therapeutic agent.

35. The method of claim 34 further comprising:
removing the frame cap of the housing of the device after said implanting;
transferring a fresh preparation of replacement cells to the cell encapsulation module of the device; and
replacing the frame cap of the housing device.

36. A method of treating diabetes in a subject, said method comprising:
implanting the cell replacement device of claim 3 into the subject having diabetes.

37. The method of claim 36, wherein the cell replacement device comprises a preparation of cells positioned in the cell encapsulation module of the device that release insulin, glucagon, or a combination for treating the subject.

38. The method of claim 37, wherein the preparation of cells comprises a preparation of islets.

39. The method of claim 38, wherein the preparation of islets is derived from a preparation of stem cells.

40. A method of treating a bleeding disorder in a subject, said method comprising:
implanting the cell replacement device of claim 3 into the subject having the bleeding disorder.

41. The method of claim 40, wherein the bleeding disorder is selected from the group consisting of hemophilia A, hemophilia B, von Willebrand disease, Factor I deficiency, Factor II deficiency, Factor V deficiency, Factor VII deficiency, Factor X deficiency, Factor XI deficiency, Factor XII deficiency, and Factor XIII deficiency.

42. A method of treating a lysosomal storage disease in a subject, said method comprising:
implanting the cell replacement device of claim 3 into the subject having the lysosomal storage disease.

43. The method of claim 42, wherein the cell replacement device comprises a preparation of cells positioned in the cell encapsulation module of the device that release an enzyme selected from α-L-iduronidase, Iduronate-2-sulfatase, α-glucuronidase, Arylsulfatase A, alpha-Galactosidase A, and combinations thereof.

44. A method of treating a cancer in a subject, said method comprising:
implanting the cell replacement device of claim 3 into the subject having cancer.

45. The method of claim 44, wherein the cell replacement device comprises a preparation of cells positioned in the cell encapsulation module of the device that release a therapeutic molecule selected from IL-2, endostatin, cytochrome P450 enzyme, tumor antigens, a cytokine, and combinations thereof.

46. A method of treating a kidney failure in a subject, said method comprising:
implanting the cell replacement device of claim 3 into the subject having a kidney failure.

47. The method of claim 46, wherein the cell replacement device comprises a preparation of cells positioned in the cell encapsulation module selected from renal proximal tubule cells, mesenchymal stem cells, and a combination thereof.

48. A method of treating a chronic pain in a subject, said method comprising:
implanting the cell replacement device of claim 3 into the subject having a chronic pain.

49. The method of claim 48, wherein the cell replacement device comprises a preparation of cells positioned in the cell encapsulation module selected from chromaffin cells, neural precursor cells, mesenchymal stem cells, astrocytes, and genetically engineered cells, or a combination thereof.

50. A cell encapsulation device kit, comprising:
plurality of different cell replacement devices, each of the plurality of different cell replacement devices comprising a frame cap, a frame base, and a mesh, wherein the frame cap includes a first connecting member and one or more ports traversing a thickness of the frame cap, wherein the frame base includes one or more walls defining an interior volume, defining a first opening to the interior volume on one side of the frame base and defining a second opening to the interior volume on another side of the frame base, the first opening configured to receive the frame cap, wherein the frame base further includes a second connecting member constructed to connect with the first connecting member, wherein at least a portion of at least one of the one or more walls is porous, and wherein the mesh is disposed adjacent the second opening; and
a plurality of different cell encapsulation modules, each of the plurality of cell encapsulation modules being configured for insertion into the interior volume of at least one of the plurality of different cell replacement devices, wherein each of the plurality of cell encapsulation modules including a nanomembrane substrate and a porous scaffold extending from the nanomembrane substrate.
